(19) United States
(12) Patent Application Publication
(10) Pub. No.: US 2021/0040550 A1
(43) Pub. Date: Feb. 11, 2021

(54) DETECTION OF NUCLEIC ACID SEQUENCES

(71) Applicant: THE UNIVERSITY OF NOTTINGHAM, NOTTINGHAM (GB)

(72) Inventors: Mohammad ILYAS, Nottingham (GB); James HASSALL, Nottingham (GB); Henry EBILI, Nottingham (GB)

(21) Appl. No.: 16/964,958

(22) PCT Filed: Jan. 25, 2019
(86) PCT No.: PCT/GB2019/050221

§ 371 Date: Jul. 24, 2020

(30) Foreign Application Priority Data
Jan. 25, 2018 (GB) 1801255.9

(51) Int. Cl. C12Q 1/6848; C12Q 1/686; C12Q 1/6858

(52) U.S. Cl. CPC C12Q 1/6848; C12Q 1/6858; C12Q 1/686

(57) ABSTRACT

The invention relates to methods of detecting a target nucleic acid having a variant sequence in a pool of nucleic acid comprising non-variant nucleic acid and/or non-targeted variant nucleic acid, and a method of highly specific PCR, together with associated primers, primers pairs, compositions, kits and uses.

This invention relates to methods of detecting a target nucleic acid having a variant sequence in a pool of nucleic acid comprising non-variant nucleic acid and/or non-targeted variant nucleic acid, and a method of highly specific PCR, together with associated primers, primers pairs, compositions, kits and uses.

Variant nucleic acids can be obtained directly from solid tissue or from bodily fluids such as plasma (also known as liquid biopsies), urine, faeces and even saliva. Unless obtained from cell lines in tissue culture, tissue-derived nucleic acids can contain a mixture of nucleic acids. For example, tumour tissue can contain nucleic acids originating from tumour cells and non-tumour cells. The former will contain mutant sequences whilst the latter, which may originate from stromal cells in a tumour or (in the case of bodily fluids) from other regions of the body, will contain wild-type (i.e. non-mutant) sequence.

The ratio of tumour cell to non-tumour cell DNA is highly variable and in certain cases (such as bodily fluids or small biopsies), the tumour cell DNA may comprise a tiny proportion of the total DNA content. In such cases, the detection of mutant alleles can become extremely problematic. The probability of finding a mutant allele is dependent on the technical performance of the assay used, and in particular, the limit-of-detection. The limit-of-detection refers to the number of variant sequences which must be present in the overall pool of nucleic acid in order for the variant sequence to be detected. For example, fluorescence-based Sanger sequencing has a limit of detection of 20%, meaning that variant sequences will not be detected in samples which contain <20% variant sequence. Previously, a number of methods have been used in order to detect low levels of variant sequence. These have ranged from the development of novel chemicals which have much greater sensitivity than standard fluorescent sequencing compounds through to enrichment of variant sequence. More recently the development of massively parallel sequencing technologies (also known as Next Generation Sequencing, NGS) has allowed detection of low level variant sequence by performing large numbers of sequencing reactions simultaneously. In addition to improved sequencing technology, screening methods have been developed, such as high resolution melting (HRM) and denaturing high purity liquid chromatography (dHPLC), which allow variant sequences to be detected at a lower limit of detection than fluorescence-based Sanger sequencing.

Currently, variant sequence detection systems which are designed to screen for variant sequence across multiple nucleotides in a single reaction use conventional PCR to amplify DNA prior to analysis. Conventional PCR maintains equal efficiency for both the variant sequence and wild-type DNA throughout a PCR program. Hence, variant sequence frequency will remain unchanged from DNA sample to PCR amplicon. To improve the sensitivity and limits of detection further for variant sequence detection systems; a solution is to modify conventional PCR to enrich specifically for the variant fraction. Variant sequence enrichment presents most benefit as a means of converting low variant sequence frequency samples into high variant sequence frequency samples. This allows the sample to become detectable as it breaches the barrier which is the limit of detection. Moreover, borderline samples which were close to the limit of detection will become more visible. I.e. if sanger sequencing was used as the variant sequence detection method for two samples which have variant sequence frequencies of 1% and 20% and conventional PCR was used to amplify them; only the 20% sample would be detected due to the 20% limit of detection. However, if an enrichment protocol was used to enrich the 1% sample to 20% and the 20% sample to over 70% then the previous 20% borderline sample is now easy to detect and the 1% sample now becomes detectable.

Wild-type blocking PCR is an enrichment system that implements a non-extendable wild-type/non-variant specific probe into the conventional PCR reaction. The probe typically overlaps 5 nucleotides with the neighbouring forward or reverse primer at the 3′ end depending on the direction chosen to inhibit with the probe. The probe is designed to have a higher annealing temperature than the primers and usually incorporates locked nucleic acids (LNAs) to increase binding affinity as well as a phosphate group on the 3′ end to prevent polymerase extension. Thus, when cycling from the denaturation temperature to the annealing temperature; the probe will bind first before the primer to the wild-type template with strong affinity; in turn preventing the primer from binding to the wild-type template. When variant sequence is present, a probe-template mismatch will occur that will cause thermodynamic instability. The mismatch instability generated will cause the probe to lose binding affinity for the variant template and bind approximately 8° C. lower for DNA-DNA mismatches and 20° C. lower for DNA-LNA mismatches. This will allow the primer to bind to the mutant template as the probe is no longer occupying the region of interest. Whilst wild-type blocking is theoretically sound, in practice, problems arise. Firstly, the probe will misprime mutant DNA and reduce its amplification efficiency. Secondly, the probes binding affinity may be weak or it may heavily mis-prime variant DNA resulting in a reduction in wild-type blocking potential and variant sequence enrichment. Thirdly, the standard annealing temperature of primers (50-60° C.) restricts the size of the probe to roughly 20-30 base pairs as a maximum gap between the annealing temperature of the probe and the primers must be 8° C. DNA-DNA mismatches are 8° C., thus, a value greater than this will begin to affect the system as the probe will bind variant DNA before the primer if its mismatched annealing temperature is greater than a perfect match primer annealing temperature.

An alternative approach to these technologies is the Amplification Refractory Mutation System (ARMS) [10]. This depends on the use of primers which are tailored for specific variant sequences. ARMS PCR based tests are more sensitive since only variant sequences undergo exponential amplification (whilst wild type sequence remains static) and, with the use of probes and real-time PCR chemistries, the sensitivity can be enhanced [11].

ARMS PCR was initially designed to test for Single Nucleotide Variants (SNVs). It works on the principle that the 3′ base of a primer needs to be bound to the template DNA in order for the polymerase enzyme to work. For example, a tumour may contain a missense mutation. In this case, the mutant and wild type sequence differ by just one base and a mutation-specific primer can be designed in which the 3′ base is complementary to the mutant sequence. If the mutation-specific primer anneals to wild type DNA, there should be no primer extension, because of the mismatch at the 3′ end. It follows from this that, in a mixture of mutant and wild type sequence, only the mutant sequence will undergo amplification and therefore, if the mutant sequence is present, it can be detected. In practice, there is usually some non-specific annealing of the mutation-specific 3′ base to wild type sequence. This then allows primer extension and results in a potential false positive. In order to improve the specificity, mismatches have been introduced [12] close to the 3′ mutation specific base but this comes at the cost of reduced efficiency and requires much more optimisation of the PCR.

An aim of the present invention is to provide improved variant sequence detection methodology and materials to be able to detect variant sequence, and in particular variant sequence in low prevalence.

According to a first aspect of the invention, there is provided a method of detecting a target nucleic acid having a variant sequence in a pool of nucleic acid comprising non-variant nucleic acid and/or non-targeted variant nucleic acid, the method comprising:
providing a forward and reverse PCR primer pair capable of hybridising to the target nucleic acid having a variant sequence for PCR amplification of the target nucleic acid having a variant sequence,
wherein the terminal 3′ nucleotide of either the forward or reverse primer is arranged to form a base pair with a variant nucleotide of interest in the target nucleic acid sequence having a variant sequence, thereby forming a variant-specific primer, and
wherein the forward and reverse PCR primers have a minimum annealing temperature (Ta) of 65° C.;
carrying out a PCR in order to amplify any target nucleic acid having a variant sequence in the pool of the nucleic acid; and
detecting any PCR product or amplification in real-time, wherein the detection of a PCR product or amplification in real-time confirms the presence of the target nucleic acid having a variant sequence in the pool of the nucleic acid.

In one embodiment, the forward and reverse PCR primers each comprise a 5′ tag of non-complementary nucleotides and/or comprise one or more nucleotide analogues such that the forward and reverse PCR primers have a minimum Ta of 65° C.

The invention advantageously takes advantage of the inherent instability of non-specific base-pairing, which is enhanced by performing PCR at a high annealing temperature (Ta). Therefore, the invention may herein be referred to as HOT ARMS PCR. The increase in free energy reduces inappropriate base-pairing between mutation-specific primer and wild-type nucleic acid thereby preventing non-specific primer extension. In contrast, perfect base matches between mutation specific primer and mutant nucleic acid are stable even at high Ta and therefore the specificity of the PCR is improved. The method does not require special probes and can be performed on standard PCR and real-time PCR machines without the need for expensive equipment. Robust mutation detection has been shown with a limit-of-detection as low as 0.004% mutant allele frequency (MAF). The method has a wide dynamic range and excellent precision even at low MAF. The test requires little or no optimisation and the tests could be multiplexed. Furthermore, it is a single stage closed-tube test which could transform cancer patient management by widening access to genetic testing. The speed of the test means it could theoretically be established in the hospital out-patient and even the primary care setting. In summary, the method provides an extremely simple, robust and sensitive test.

Although basic ARMS PCR is known to be used for SNV detection, the present invention can be applied to any situation where a novel sequence is created by juxtaposition of pre-existing sequences (such as DNA rearrangement or DNA deletion) or by insertion of novel sequences adjacent to pre-existing sequence (such as DNA insertion or DNA amplification). In this case, the primer can be designed in which all bases are complementary to the wild-type sequence apart from the 3′ base which is complementary to the novel juxtaposed sequence. The principle of the mismatched 3′ base will preventing primer extension (and thereby preventing PCR) will apply in the same way as for a SNV detection.

According to another aspect of the present invention there is provided a method of detecting a target nucleic acid containing variant sequence in a pool of wild-type nucleic acid, the method comprising:
providing a forward and reverse PCR primer pair capable of hybridising to the variant target nucleic acid for PCR amplification of the variant-containing target nucleic acid,
wherein the terminal 3′ nucleotide of either the forward or reverse primer is
arranged to form a base pair with the mutation of interest in the mutation-containing target nucleic acid sequence, and
wherein the forward and reverse PCR primers have a minimum Ta of 65° C.;
carrying out a PCR in order to amplify any variant-containing target nucleic acid in the pool of the target nucleic acid; and
detecting any PCR product or amplification in real-time, wherein the detection of a PCR product or amplification in real-time confirms the presence of the variant-containing target nucleic acid in the pool of the target nucleic acid; and
optionally wherein the forward and reverse PCR primers each comprise a 5′ tag of non-complementary nucleotides and/or comprise one or more nucleotide analogues such that the forward and reverse PCR primers have a minimum Ta of 65° C.

The non-variant nucleic acid may be wild-type nucleic acid. The term “variant” or “variant sequence” when used in the context of a nucleic acid sequence may be used to refer to a nucleic acid that is different in sequence to a more common and/or normal nucleic acid that may be present in a pool of nucleic acids. Moreover, wild-type and/or normal sequence may be known as the sequence which is considered normal and/or common for the pool nucleic acids. Therefore, a variant may comprise a nucleic acid sequence that is different in sequence relative to the sequence of another nucleic acid, which may be termed “non-variant” or “wild-type”, in the pool of nucleic acids.

The non-variant nucleic acid may be present in the pool of nucleic acid in greater proportion than the variant nucleic acid. In one embodiment, the pool of nucleic acid may comprise at least 10-fold less variant nucleic acid relative to non-variant nucleic acid. In another embodiment, the pool of nucleic acid may comprise at least 50-fold less variant nucleic acid relative to non-variant nucleic acid. In another embodiment, the pool of nucleic acid may comprise at least 100-fold less variant nucleic acid relative to non-variant nucleic acid. In another embodiment, the pool of nucleic acid may comprise at least 500-fold less variant nucleic acid relative to non-variant nucleic acid. In another embodiment, the pool of nucleic acid may comprise at least 1000-fold less variant nucleic acid relative to non-variant nucleic acid. In another embodiment, the pool of nucleic acid may comprise at least 5000-fold less variant nucleic acid relative to non-variant nucleic acid. In another embodiment, the pool of nucleic acid may comprise at least 10000-fold less variant nucleic acid relative to non-variant nucleic acid. In another embodiment, the pool of nucleic acid may comprise at least 50000-fold less variant nucleic acid relative to non-variant nucleic acid. In another embodiment, the pool of nucleic acid may comprise less than 10,0000-fold less variant nucleic acid relative to non-variant nucleic acid. In another embodiment, the pool of nucleic acid may comprise less than 50000-fold less variant nucleic acid relative to non-variant nucleic acid.

The pool of nucleic acid may comprise a plurality of nucleic acids that have minor sequence differences, whereby the intention is to detect and/or enrich a targeted nucleic acid having a variant sequence and not other similar sequences that may be present in the pool of nucleic acid. For example, a pool of nucleic acids derived from various strains of microorganism may have similar but diverse sequences, but the sequence of only one of the strains may be of interest for detection or enrichment. Therefore, the variant nucleic acid sequence of interest may be considered the “targeted nucleic acid having a variant sequence”, and the remaining nucleic acid sequences in the pool of nucleic acid may be considered “non-targeted variant nucleic acid”.

The pool of nucleic acid may be in a sample, such as a sample from a subject. The sample may comprise a cell lysate, a bodily fluid sample, or a nucleic acid sample, such as a sample of purified or partially purified nucleic acid. The sample may be cell free. The bodily fluid sample may be blood, blood serum or mucous, such as saliva. The pool of nucleic acid may comprise a plurality of nucleic acid sequences. The pool of nucleic acid may comprise mutation-containing target nucleic acid and the wild-type variant of the target nucleic acid (i.e. not containing a mutation). The nucleic acid may be DNA or RNA, or a mixture thereof. The pool of nucleic acid may comprise maternal and foetal nucleic acid, wherein the foetal nucleic acid may be the targeted nucleic acid having the variant sequence relative to the maternal nucleic acid.

In another embodiment, the pool of nucleic acid may be from a single cell or a population of cells. The cell or population of cells may be eukaryote or prokaryote. The cell or population of cells may be mammalian or fungal. The cell or population of cells may be human. The cell, population of cells, or sample may be derived from a patient. For example, a patient having a condition, or suspected of having a condition, or at risk of having a condition. The cell, population of cells, or sample may be derived from a patient of unknown condition. The target nucleic acid, cell or population of cells may be from a subject who has, or is suspected to have, or is at risk of having, a condition associated with a mutation or variation. The mutation or variation may be associated with a disease or condition. The mutation may be indicative of a disease or condition. The indication may be diagnostic, prognostic or predictive of response to therapy. The indication may be an indication of risk or likelihood of developing a disease or condition. Such conditions may comprise cancer of any type including cancers arising from epithelium (such as lung adenocarcinoma, ductal carcinoma of the pancreas, breast cancer or colorectal cancer), tumours arising from the mesenchyme (such as sarcoma), cancer arising from the haemopoietic or lymphoid tissue (such as lymphoma or leukaemia) and cancers arising from gonads (such as testicular cancers and ovarian cancers). The condition may be selected from Cystic Fibrosis, Neurofibromatosis, Sickle-Cell Anemia, Tay-Sachs disease. Additionally or alternatively, the condition may be selected from Fabry disease, Phenylketonuria, Siderius type X-linked mental retardation, N-Glycanase 1 deficiency, Fibrodysplasia ossificans progressive, Polygamist Down's, Biotinidase deficiency, 3-hydroxy-3-methylglutaryl-CoA lyase deficiency, pyruvate dehydrogenase deficiency, Leigh disease, Lesch-Nyhan syndrome, Ogden syndrome, Gaucher's disease, 3-Methylcrotonyl-CoA carboxylase deficiency, Methyl-CoA mutase deficiency. The skilled person will understand that the present invention can be used to test any cancer or syndrome where the mutation is known or deducible. A skilled person will be able to design an assay with a primer according to the invention for the mutation or variant of interest.

In one embodiment, the target nucleic acid having a variant sequence may be eukaryote, prokaryote or viral nucleic acid. The eukaryote nucleic acid may be mammalian or fungal nucleic acid. In one embodiment the target nucleic acid having a variant sequence is human. The target nucleic acid having a variant sequence may be associated with a disease or condition. The target nucleic acid having a variant sequence may comprise or consist of DNA or RNA. The target nucleic acid having a variant sequence may comprise genomic nucleic acid. The target nucleic acid having a variant sequence may comprise viral RNA; mRNA; ncRNA; miRNA; and siRNA; or combinations thereof. The target nucleic acid having a variant sequence may comprise mitochondrial nucleic acid. The target nucleic acid having a variant sequence may comprise or consist of chromosomal and/or non-chromosomal DNA. In one embodiment, the target nucleic acid having a variant sequence acid comprises circulating DNA, such as circulating tumour DNA (ctDNA). In one embodiment, the target nucleic acid having a variant sequence comprises mRNA transcript.

The variant sequence or variant nucleotide may represent a sequence change such that a new sequence is created which is different from the wild-type sequence or non-variant sequence. In one embodiment the variant sequence or variant nucleotide comprises a single nucleotide variation (SNV) whereby there is sequence variation of a single nucleotide to an alternative nucleotide. The nucleotide that is subject to a variation may comprise adenine (A), thymine (T), cytosine (C), or guanine (G), or in the case of RNA, adenine (A), uracil (U), cytosine (C), or guanine (G). In another embodiment, the variant sequence comprises a new sequence generated due to nucleotide deletion. In this embodiment, irrespective of the number of nucleotides deleted, the nucleic acid sequence just before the deleted region is brought into juxtaposition with the nucleic acid sequence just after the deleted region thereby creating a new sequence. Where there is a nucleotide deletion, the 3′ nucleotide of the mutation specific primer (either the forward or reverse primer) is arranged to base pair with the next downstream nucleotide following the deleted nucleotide(s) in the target sequence. Thus, all nucleotides in the variant-specific primer will be paired with bases in the wild-type nucleic acid except for the 3′ nucleotide which will pair with the newly juxtaposed sequence.

In an embodiment wherein the variant sequence is a nucleotide insertion or a nucleotide amplification, a new sequence is generated by addition of new nucleotides into the wild-type sequence. In this embodiment, the 3′ nucleotide of either the forward or reverse primer that is arranged to base pair with the variant sequence of interest may be arranged to base pair with the added nucleotide. Thus, all nucleotides in the variant-specific primer will be paired with bases in the wild-type nucleic acid except for the 3′ nucleotide which will pair with the newly inserted sequence.

In an embodiment wherein the variant sequence is a nucleotide rearrangement (such as a chromosomal translocation or inversion), a new sequence is generated by juxtaposition of nucleotides from differing genomic region. In this embodiment, the 3′ nucleotide of either the forward or reverse primer that is arranged to base pair with the mutation of interest may be arranged to base pair with the re-arranged nucleotide. Thus, all nucleotides in the mutation-specific primer will be paired with bases located in one region except for the 3′ nucleotide which will pair with the nucleotide belonging to the newly juxtaposed region.

The forward and/or reverse primers may comprise or consist of DNA. Additionally or alternatively, the forward and/or reverse primers may comprise a nucleotide analogue or derivative, such as a functional nucleotide analogue or derivative having equivalent complementation as DNA or RNA. The forward and/or reverse primers may comprise combinations of DNA, RNA and/or nucleotide analogues. In particular, the skilled person will understand that the primer could contain a tag, or nucleotide analogue(s), or a combination of both. The number of nucleotide analogues could range from 1 to any number that is required to provide a Ta≥65° C. Any combination of these features may be provided for the primer provided that the Ta is ≥65° C. Nucleotide analogues may comprise or consist of Locked Nucleic Acids (LNA), Bridged Nucleic Acids (BNA) and Peptide Nucleic Acids (PNA) or other nucleotide analogues which enhance primer binding specificity and/or increase annealing temperature of oligonucleotides). In one embodiment the forward or the reverse primer may comprise a nucleotide analogue, such as LNA, PNA, or BNA, at the terminal 3′ position. In one embodiment the forward or the reverse primer may comprise a nucleotide analogue, such as LNA, PNA, or BNA, at the terminal 3′ position, wherein the remaining primer sequence comprises DNA. In another embodiment the forward or the reverse primer may comprise a nucleotide analogue, such as LNA, PNA, or BNA, at the terminal 3′ position, and a 5′ tag of non-complementary nucleotides.

The use of a nucleotide analogue, particularly in the terminal 3′ position, advantageously provides a more stringent annealing and PCR. The nucleotide analogue can raise the Tm/Ta of the primer.

In one embodiment, the terminal 3′ nucleotide of the forward primer is arranged to form a base pair with the variant sequence of interest in the target nucleic acid having a variant sequence. Alternatively, the terminal 3′ nucleotide of the reverse primer is arranged to form a base pair with the variant sequence of interest in the target nucleic acid having a variant sequence. The skilled person may choose either the forward or reverse primer as the variant sequence binding member of the pair depending on which is the most efficient for a given sequence. For Example, for the KRAS, PIK3CA, EGFR, APC or BRAF mutation described herein, the terminal 3′ nucleotide of the reverse primer may be arranged to form a base pair with the variant sequence of interest in the target nucleic acid having a variant sequence.

The skilled person will understand that any suitable lengths of forward and reverse primer may be used, and may be designed according to the needs of any particular target sequence. However, certain length of primer may be more of an optimum than others for the hybridisation and PCR. In one embodiment, the forward and/or reverse primers may be at least about 15 nucleotides in length. In one embodiment, the forward and/or reverse primers may be at least about 20 nucleotides in length. In another embodiment, the forward and/or reverse primers may be at least about 24 nucleotides in length. The forward and/or reverse primers may be no more than about 40 nucleotides in length. The forward and/or reverse primers may be no more than about 35 nucleotides in length. The forward and/or reverse primers may be no more than about 30 nucleotides in length. In another embodiment, the forward and/or reverse primers may be no more than about 26 nucleotides in length. In another embodiment, the forward and/or reverse primers may be, or may be no more than, about 42 nucleotides in length. In another embodiment, the forward and/or reverse primers may be no more than about 50 nucleotides in length. In another embodiment, the forward and/or reverse primers may be no more than about 60 nucleotides in length. In one embodiment, the forward and/or reverse primers may be between about 15 and about 60 nucleotides in length. In one embodiment, the forward and/or reverse primers may be between about 15 and about 50 nucleotides in length. In one embodiment, the forward and/or reverse primers may be between about 15 and about 40 nucleotides in length. In another embodiment, the forward and/or reverse primers may be between about 15 and about 35 nucleotides in length. In one embodiment, the forward and/or reverse primers may be between about 20 and about 30 nucleotides in length. In another embodiment, the forward and/or reverse primers may be between about 22 and about 28 nucleotides in length. In another embodiment, the forward and/or reverse primers may be between about 24 and about 26 nucleotides in length. In another embodiment, the forward and/or reverse primers may be about 25 nucleotides in length. Reference to the primer length herein excludes any 5′ tag (e.g. 5′ GC rich tag).

The skilled person will recognise that a primer can be provided with a higher Ta by increasing its length, as an alternative or in addition to, providing a 5′ non-complementary tag and/or including nucleotide analogues. In particular, primers may be designed to be longer than usual to obtain at least 65° C. Ta through length alone. Although, a preferred option to provide a Ta of at least 65° C. is to modify the primers by attaching a 5′ non-complementary tag and/or including nucleotide analogues.

One advantage of 5′ tags is that they provide a very large increase to the annealing temperature (for example, +6° C. as standard and potentially up to +15° C.); more so than modified bases (+1 to +4° C.). The 5′ tags also have a second benefit as they improve selectivity and reduce PCR cycling time. When tagged primers initially bind DNA the tag only partially binds as DNA does not contain the tag sequence. When primers form amplicons they are incorporated. Thus, when amplicons are formed, the tag sequence is present in the template for primers to bind with perfect complementarity rather than partial and this causes further gains in annealing temperature as well as selectivity for mutant DNA amplicons early on before non-specific product generation occurs in exponential amplification. This novel finding generates the ability to generate a 2 phase cycling PCR with phase 1 having a lower annealing temperature than phase 2. During phase 2 the raised annealing temperature will provide preferential amplification of amplicons rather than DNA and reduced cycling times. For example, in cycle 1 as amplicons are forming the annealing temperature may be 71° C. After cycle 1 with tagged primers the annealing temperature can be increased for example to 72-80° C. 10 cycles are recommended for the lower annealing temperature to assure amplicon formation. Since polymerase activity is between 68-80° C., extension can still occur. Cycling now alternates as 2 steps between 95° C. and 75-80° C. rather than a standard 3 step protocol alternating between 95° C., 60° C. and 72° C. or a 2-step protocol alternating between 95° C. and 60° C. Compared to standard PCR protocols, tag incorporated primers reduce the amount of time ramping which in turn reduces the overall PCR time.

The forward and reverse primers may comprise a known/pre-determined sequence. The forward and reverse primers may be respectively complementary to the sense and anti-sense strands of the target nucleic acid sequence. The skilled person will understand that one or two, or more, mismatches may be present in the primer sequences and the primers can still function, although with less efficiency. Therefore, the primers may or may not be 100% complementary to the sense/anti-sense strand, but they should be sufficiently complementary to function as primers. In an embodiment wherein the forward primer contains the terminal 3′ nucleotide arranged to base pair with the mutation of interest, the reverse primer may comprise or consist of wild-type sequence relative to the target nucleic acid. In an embodiment wherein the reverse primer contains the terminal 3′ nucleotide arranged to base pair with the variant nucleotide of interest, the forward primer may comprise or consist of wild-type sequence relative to the target nucleic acid. In one embodiment the forward and/or reverse primers are variant-specific primers.

The forward and reverse primers may be arranged to amplify a PCR product of at least about 60 bp in length. The forward and reverse primers may be arranged to amplify a PCR product of 110 bp or less in length. The forward and reverse primers may be arranged to amplify a PCR product of between about 60 and about 110 bp in length. In another embodiment, the forward and reverse primers may be arranged to amplify a PCR product of between about 60 and about 100 bp in length. In another embodiment, the forward and reverse primers may be arranged to amplify a PCR product of between about 60 and about 80 bp in length. In another embodiment, the forward and reverse primers may be arranged to amplify a PCR product of up to about 20000 bp in length. In another embodiment, the forward and reverse primers may be arranged to amplify a PCR product of between about 60 and about 20000 bp in length. In another embodiment, the forward and reverse primers may be arranged to amplify a PCR product of between about 60 and about 10000 bp in length. In another embodiment, the forward and reverse primers may be arranged to amplify a PCR product of between about 60 and about 5000 bp in length. In another embodiment, the forward and reverse primers may be arranged to amplify a PCR product of between about 60 and about 1000 bp in length.

The PCR product size of between about 60 and about 110 bp in length is favourable for detection of mutations, particularly in formalin-fixed tissue, where the nucleic acid is often degraded. The PCR product size of up to 20000 bp is beneficial for plasmids, such as in plasmid based site directed mutagenesis.

In one embodiment, the forward and/or reverse primers may comprise a GC content of at least 5%. In one embodiment, the forward and/or reverse primers may comprise a GC content of at least 10%. In one embodiment, the forward and/or reverse primers may comprise a GC content of at least 20%. In one embodiment, the forward and/or reverse primers may comprise a GC content of at least 30%. In another embodiment, the forward and/or reverse primers may comprise a GC content of at least 35%. In another embodiment, the forward and/or reverse primers may comprise a GC content of at least 40%. In another embodiment, the forward and/or reverse primers may comprise a GC content of at least 42%. In another embodiment, the forward and/or reverse primers may comprise a GC content of 60% or less. In another embodiment, the forward and/or reverse primers may comprise a GC content of 55% or less. In another embodiment, the forward and/or reverse primers may comprise a GC content of 50% or less. In another embodiment, the forward and/or reverse primers may comprise a GC content of 48% or less. In another embodiment, the forward and/or reverse primers may comprise a GC content of between about 5% and about 95%. In another embodiment, the forward and/or reverse primers may comprise a GC content of between about 10% and about 60%. In another embodiment, the forward and/or reverse primers may comprise a GC content of between about 20% and about 60%. In another embodiment, the forward and/or reverse primers may comprise a GC content of between about 40% and about 60%. In another embodiment, the forward and/or reverse primers may comprise a GC content of between about 45% and about 60%. In another embodiment, the forward and/or reverse primers may comprise a GC content of between about 40% and about 55%. In another embodiment, the forward and/or reverse primers may comprise a GC content of between about 40% and about 50%. In another embodiment, the forward and/or reverse primers may comprise a GC content of between about 42% and about 48%. In another embodiment, the forward and/or reverse primers may comprise a GC content of about 45%. Reference to the GC content of forward and/or reverse primers is intended to refer to the GC content of the primers alone and the calculation does not including any 5′ tag GC content.

Advantageously, providing a GC rich sequence ensures that the melting temperature (Tm) of the primer is increased, in order to allow the PCR to be performed at a higher annealing temperature (Ta). The high Ta makes for a more stringent reaction condition thereby improving the specificity.

In one embodiment, the forward and/or reverse primers (optionally modified by attaching a 5′ non-complementary tag and/or including nucleotide analogues) may have a Tm (melting temperature) of 65° C. or more. In another embodiment, the forward and/or reverse primers may have a Tm of at least 66° C., 67° C., or 68° C. In another embodiment, the forward and/or reverse primers may have a Tm of 85° C. or less. In another embodiment, the forward and/or reverse primers may have a Tm of 80° C. or less. In another embodiment, the forward and/or reverse primers may have a Tm of 75° C. or less. In another embodiment, the forward and/or reverse primers may have a Tm of 70° C. or less. In another embodiment, the forward and/or reverse primers may have a Tm of 68° C. or less. In another embodiment, the forward and/or reverse primers may have a Tm of between about 65° C. and about 90° C. In another embodiment, the forward and/or reverse primers may have a Tm of between about 65° C. and about 99° C. In another embodiment, the forward and/or reverse primers may have a Tm of between about 65° C. and about 75° C. In another embodiment, the forward and/or reverse primers may have a Tm of between about 65° C. and about 85° C. In another embodiment, the forward and/or reverse primers may have a Tm of between about 65° C. and about 70° C.

In one embodiment, the forward and/or reverse primers (optionally modified by attaching a 5′ non-complementary tag and/or including nucleotide analogues) may have a Ta (annealing temperature) of 65° C. In another embodiment, the forward and/or reverse primers may have a Ta (annealing temperature) of greater than 65° C. In another embodiment, the forward and/or reverse primers may have a Ta of at least 66° C., 67° C., or 68° C. In another embodiment, the forward and/or reverse primers may have a Ta of 85° C. or less. In another embodiment, the forward and/or reverse primers may have a Ta of 80° C. or less. In another embodiment, the forward and/or reverse primers may have a Ta of 75° C. or less. In another embodiment, the forward and/or reverse primers may have a Ta of 70° C. or less. In another embodiment, the forward and/or reverse primers may have a Ta of between about 65° C. and about 85° C. In another embodiment, the forward and/or reverse primers may have a Ta of between about 65° C. and about 99° C. In another embodiment, the forward and/or reverse primers may have a Ta of between about 65° C. and about 75° C. In another embodiment, the forward and/or reverse primers may have a Ta of between about 65° C. and about 85° C.

The Tm difference between the forward and reverse primer may be about 2° C. or less. In another embodiment, the Tm difference between the forward and reverse primer may be about 3° C. or less. Greater differences will still allow the PCR to work but there may be a loss of efficiency. For example, the Tm difference between the forward and reverse primer may be about 4° C. or less. In another embodiment, the Tm difference between the forward and reverse primer may be about 5° C. or less. In another embodiment, the Tm difference between the forward and reverse primer may be about 6° C. or less. In another embodiment, the Tm difference between the forward and reverse primer may be about 8° C. or less. In another embodiment, the Tm difference between the forward and reverse primer may be about 10° C. or less. In another embodiment, the Tm difference between the forward and reverse primer may be about 15° C. or less. In another embodiment, the Tm difference between the forward and reverse primer may be about 20° C. or less.

The skilled person will understand that primers designed with a high Tm can have a very wide range of annealing; such as at least 20° C. The 5′ tag aids this so a wider gap between the forward and reverse primer will be more acceptable than a standard primer pair without 5′ tag.

The forward or reverse primer may comprise the sequence of any of SEQ ID NOs: 1-6, 8, 10, 12, 14 or 16.

The 5′ tag of non-complementary nucleotides may consist of a sequence of up to 100 nucleotides. The 5′ tag of non-complementary nucleotides may consist of a sequence of between about 5 and 100 nucleotides. In another embodiment, the 5′ tag of non-complementary nucleotides may consist of a sequence of between about 5 and 80 nucleotides. In another embodiment, the 5′ tag of non-complementary nucleotides may consist of a sequence of between about 5 and 60 nucleotides. In another embodiment, the 5′ tag of non-complementary nucleotides may consist of a sequence of between about 5 and 50 nucleotides. In another embodiment, the 5′ tag of non-complementary nucleotides may consist of a sequence of between about 5 and 30 nucleotides. In another embodiment, the 5′ tag of non-complementary nucleotides may consist of a sequence of between about 5 and 25 nucleotides. The 5′ tag of non-complementary nucleotides may consist of a sequence of between about 5 and 15 nucleotides. In another embodiment, the 5′ tag of non-complementary nucleotides may consist of a sequence of between about 8 and about 100 nucleotides. In another embodiment, the 5′ tag of non-complementary nucleotides may consist of a sequence of between about 8 and about 50 nucleotides. In another embodiment, the 5′ tag of non-complementary nucleotides may consist of a sequence of between about 8 and about 15 nucleotides. In another embodiment, the 5′ tag of non-complementary nucleotides may consist of a sequence of between about 10 and about 100 nucleotides. In another embodiment, the 5′ tag of non-complementary nucleotides may consist of a sequence of between about 10 and about 50 nucleotides. In another embodiment, the 5′ tag of non-complementary nucleotides may consist of a sequence of between about 10 and about 15 nucleotides. In another embodiment, the 5′ tag of non-complementary nucleotides may consist of a sequence of between about 10 nucleotides. The 5′ tag of non-complementary nucleotides preferably comprises a sequence comprised of G and C nucleotides. The GC content of the 5′ tag may be 100%. The skilled person will recognise that other nucleotides can be included in the tag but are likely to be less effective in raising the Ta of the PCR. Therefore, in one embodiment, the 5′ tag of non-complementary nucleotides preferably comprises a sequence consisting of G and C nucleotides. In one embodiment, at least 90% of nucleotides of the 5′ tag of non-complementary nucleotides are G or C nucleotides. In another embodiment, at least 80% of nucleotides of the 5′ tag of non-complementary nucleotides are G or C nucleotides. In another embodiment, at least 70% of nucleotides of the 5′ tag of non-complementary nucleotides are G or C nucleotides.

The 5′ tag of non-complementary nucleotides may comprise or consist of a sequence of 5′-gggccggccc-3′ (SEQ ID NO: 35) or 5′-gggccgggccggccc-3′(SEQ ID NO: 36). Ordinarily such sequences will suffice but in some instances the skilled person will recognise that the G and C nucleotides may be arranged in a different order, for example due to constraints imposed by nearest-neighbour effects of nucleic acid sequence adjacent to the mutation specific primer. In one embodiment, the forward or reverse primer contains a 5′ tag of non-complementary GC nucleotides together with a nucleotide analogue, such as LNA, incorporated as the terminal 3′ nucleotide, which is specific for the mutation.

The method may, in addition, contain a second primer pair targeted to a different nucleic acid sequence as a control to positively verify the PCR is working and confirm that a negative result is due to an absence of the mutation-containing target nucleic acid rather than a failed PCR.

The skilled person will readily understand and provide the necessary reagent and conditions for the PCR cycles. In particular, the PCR may comprise the use of a polymerase which does not have proof-reading activity to amplify the target nucleic acid and for the generation of mutation specific PCR product.

In one embodiment, the annealing temperatures used in the PCR cycles will be at least 65° C. or more due to the addition of the GC-tags. The skilled person will be able to match an annealing temperature to the predicted melting temperatures of the tagged primers. The skilled person will understand that the Ta is usually 5° C. lower that the melting temperature (Tm) and may empirically find the optimal Ta for tagged primers. In one embodiment, an annealing temperature of about 71° C. is used.

Advantageously, an annealing temperature of about 71° C. can be used as this will work in most cases without needing any further optimization.

The PCR may be run for at least 30 cycles and can be extended to 50 cycles. In another embodiment, the PCR may be run for at least 3 cycles and can be extended to 100 cycles. In most cases, 40 cycles will suffice for the detection of a single copy of mutation-containing target nucleic acid.

In one embodiment, at least about 6 mM magnesium is used in the PCR reaction. In another embodiment, between about 1 mM and about 7 mM magnesium is used in the PCR reaction. In another embodiment, at least about 6 mM magnesium is used in the PCR reaction. In another embodiment, between about 0.1 mM and about 7 mM magnesium is used in the PCR reaction. In another embodiment, between about 1 mM and about 6 mM magnesium is used in the PCR reaction. In another embodiment, between about 3 mM and about 7 mM magnesium is used in the PCR reaction. In another embodiment, between about 4 mM and about 6 mM magnesium is used in the PCR reaction. In another embodiment, about 6 mM magnesium is used in the PCR reaction.

The amplification of the PCR product may be detected during the PCR, for example if a Real-Time PCR machine is used. This advantageously may not require any further test to be performed. Additionally or alternatively, for example if a Real-Time PCR machine is not used, the amplification of the PCR product may be detected by performing a supplementary end-point test (i.e. after completion of the PCR). Such tests include gel-electrophoresis or high resolution melting of the PCR products. The skilled person will be familiar with a range of techniques and methods for detecting, measuring and visualizing PCR product.

The method may further comprise the provision of two or more (i.e. a plurality of) variant specific primer pairs that are arranged to target a different target nucleic acid having a variant sequence, or a different variant sequence of the target nucleic acid having a variant sequence relative to each other.

According to another aspect of the invention, there is provided a method of determining the status of a condition associated with a known variant sequence in a subject, the method comprising:
providing a sample obtained from the subject comprising a nucleic acid, wherein the nucleic acid may contain a target nucleic acid having a variant sequence;
detecting the target nucleic acid having a variant sequence in the sample in accordance with the method of the invention herein,
wherein the detection of the target nucleic acid having a variant sequence is indicative of the status of the condition associated with the variant sequence in the subject.

In one embodiment, the status may provide a diagnosis or prognosis for the condition, or may provide information on the therapy choices of the subject. Additionally or alternatively, the status may comprise the progression of the condition. Further additionally or alternatively, the status may inform on the severity of the condition.

The condition may comprise cancer of any type, including cancers arising from epithelium (such as lung adenocarcinoma, ductal carcinoma of the pancreas, breast cancer or colorectal cancer), tumours arising from the mesenchyme (such as sarcoma), cancer arising from the haemopoietic or lymphoid tissue (such as lymphoma or leukaemia) and cancers arising from gonads (such as testicular cancers and ovarian cancers). The condition may comprise any disease or condition associated with a variant sequence, such as a mutation.

According to another aspect of the present invention, there is provided a primer for use in a primer pair for detecting a target nucleic acid having a variant sequence in a pool of nucleic acid comprising non-variant nucleic acid and/or non-targeted variant nucleic acid, wherein the primer is capable of hybridising to the target nucleic acid having a variant sequence for PCR amplification of the target nucleic acid having a variant sequence, wherein the terminal 3′ nucleotide of the primer is arranged to base pair with a variant nucleotide of interest in the target nucleic acid having a variant sequence; wherein the primer comprises a 5′ tag of non-complementary nucleotides and/or comprise one or more nucleotide analogues such that the primers has a minimum Ta of 65° C.

According to another aspect of the present invention, there is provided a primer for use in a primer pair for detecting a target nucleic acid having a variant sequence,
wherein the primer comprises a 5′ tag of non-complementary nucleotides and/or comprise one or more nucleotide analogues such that the primers has a minimum Ta of 65° C.

In one embodiment, the primer comprises a nucleotide analogue, such as LNA, PNA, or BNA at any position. In one embodiment, the primer comprises a nucleotide analogue, such as LNA, PNA, or BNA, at any position, and DNA for the remaining nucleotide positions. In one embodiment, the primer comprises a nucleotide analogue, such as LNA, PNA, or BNA, at the terminal 3′ position. In one embodiment, the primer comprises a nucleotide analogue, such as LNA, PNA, or BNA, at the terminal 3′ position, and DNA for the remaining nucleotide positions. In one embodiment, the primer comprises a nucleotide analogue, such as LNA, PNA, or BNA, at the terminal 3′ position, and a 5′ tag of non-complementary nucleotides. The skilled person will recognise that any suitable nucleotide analogue may be used that can increase specificity and/or increase annealing temperatures of a primer or probe. Therefore, the nucleotide analogue may comprise a nucleotide analogue that is capable of increase annealing temperature of a primer relative to an analogous A, C, T or G nucleotide.

The features of the primer(s) according to the method of the invention herein may equally apply as embodiments or aspects of primer for use in a primer pair according to the invention described herein.

According to another aspect of the present invention, there is provided a forward and reverse primer pair for the PCR detection of a KRAS mutation in a target nucleic acid, which is related to cancer, wherein the forward primer comprises the sequence of any of forward KRAS primer described in Table 1 herein, and the reverse primer comprises the sequence of any reverse KRAS primer described in Table 1 herein, and
wherein the forward and reverse primers further comprise a 5′ tag of nucleotides that are non-complementary to the target nucleic acid, and/or wherein terminal 3′ nucleotide of the forward or reverse primer is substituted with a nucleotide analogue.

The cancer related to the KRAS mutation may be colorectal cancer.

According to another aspect of the present invention, there is provided a forward and reverse primer pair for the PCR detection of a PIK3CA mutation in a target nucleic acid, which is related to cancer, wherein the forward primer comprises the sequence of any forward PIK3CA primer described in Table 1 herein, and the reverse primer comprises the sequence of any reverse PIK3CA primer described in Table 1 herein, and
wherein the forward and reverse primers further comprise a 5′ tag of nucleotides that are non-complementary to the target nucleic acid, and/or wherein terminal 3′ nucleotide of the forward or reverse primer is substituted with a nucleotide analogue.

According to another aspect of the present invention, there is provided a forward and reverse primer pair for the PCR detection of a APC mutation in a target nucleic acid, which is related to cancer, wherein the forward primer comprises the sequence of any forward APC primer described in Table 1 herein, and the reverse primer comprises the sequence of any reverse APC primer described in Table 1 herein, and
wherein the forward and reverse primers further comprise a 5′ tag of nucleotides that are non-complementary to the target nucleic acid, and/or wherein terminal 3′ nucleotide of the forward or reverse primer is substituted with a nucleotide analogue.

According to another aspect of the present invention, there is provided a forward and reverse primer pair for the PCR detection of a BRAF mutation in a target nucleic acid, which is related to cancer, wherein the forward primer comprises the sequence of any forward BRAF primer described in Table 1 herein and the reverse primer comprises the sequence of any reverse BRAF primer described in Table 1 herein, and
wherein the forward and reverse primers further comprise a 5′ tag of nucleotides that are non-complementary to the target nucleic acid, and/or wherein terminal 3′ nucleotide of the forward or reverse primer is substituted with a nucleotide analogue.

According to another aspect of the present invention, there is provided a forward and reverse primer pair for the PCR detection of a EGFR mutation in a target nucleic acid, which is related to cancer, wherein the forward primer comprises the sequence of any forward EGFR primer described in Table 1 herein and the reverse primer comprises the sequence of any reverse EGFR primer described in Table 1 herein, and
wherein the forward and reverse primers further comprise a 5′ tag of nucleotides that are non-complementary to the target nucleic acid, and/or wherein terminal 3′ nucleotide of the forward or reverse primer is substituted with a nucleotide analogue.

According to another aspect of the invention, there is provided a composition comprising the primer or primer pair according to the present invention.

The composition may further comprise a blocking probe as described herein.

The composition may comprise a plurality (i.e. two or more) of sets of different primer pairs that are arranged to PCR amplify different target sequences.

According to another aspect of the invention, there is provided a kit comprising the primer pair according to the present invention.

The kit may comprise both forward and reverse primers of the primer pair. The kit may comprise a plurality (i.e. two or more) of sets of different primer pairs that are arranged to PCR amplify different target sequences. The kit may comprise a polymerase.

The kit may further comprise a blocking probe as described herein.

According to another aspect of the invention, there is provided the use of the primer, primer pair, composition or kit in accordance with the invention herein, for the detection of a target nucleic acid having a variant sequence in a pool of nucleic acid comprising non-variant nucleic acid and/or non-targeted variant nucleic acid.

According to another aspect of the invention, there is provided the use of the primer, primer pair, composition or kit in accordance with the invention herein, for diagnosis or prognosis of a condition or response to chemotherapy associated with a mutation, for example in a tumour, of a subject.

The variant/mutation may be associated with any of KRAS, PIK3CA, EGFR, APC or BRAF. The diagnosis or prognosis may comprise detecting a variation or mutation in KRAS, PIK3CA, EGFR, APC or BRAF. The diagnosis or prognosis may be for conditions or diseases associated with a mutation in KRAS, PIK3CA, EGFR, APC or BRAF.

Probe Inhibited PCR

According to another aspect of the invention, there is provided a method of detecting a target nucleic acid having a variant sequence in a pool of nucleic acid that comprises non-variant nucleic acid and/or non-targeted variant nucleic acid, the method comprising:
i) providing a forward and reverse PCR primer pair capable of hybridising to the target nucleic acid having a variant sequence for PCR amplification of the target nucleic acid having a variant sequence, wherein the forward and reverse PCR primers have a minimum Ta of 65° C.;
ii) providing a blocking probe that is arranged to hybridise to the variant nucleic acid and/or non-targeted variant nucleic acid and prevent polymerisation from the forward and/or reverse PCR primers;
carrying out a PCR in order to amplify any target nucleic acid having a variant sequence in the pool of the nucleic acid; and
detecting any PCR product or amplification in real-time, wherein the detection of a PCR product or amplification in real-time confirms the presence of the target nucleic acid having a variant sequence in the pool of the nucleic acid.

In one embodiment of the aspect of the invention requiring a blocking probe (HOT PI PCR), the forward and reverse PCR primers each comprise a 5′ tag of non-complementary nucleotides and/or comprise one or more nucleotide analogues such that the forward and reverse PCR primers have a minimum Ta of 65° C. The forward and reverse PCR primer pair may be capable of hybridising to the non-variant sequence, such as wild-type sequence, and/or non-targeted variant sequence(s).

The invention advantageously counteracts issues of standard wild-type blocking/probe inhibited PCR by increasing the primer annealing temperature (Ta), for example by using primer modifications and/or longer primers. The increase in free energy generated by raising the annealing temperature to >65° C. can prevent inappropriate base-pairing and perfect base matches would be more stable resulting in the ability to provide a blocking probe which had higher specificity. Hence, giving more freedom for the mutant DNA to amplify without probe mispriming and blocking the wild-type DNA further as the probe would bind with greater affinity. Moreover, raising the annealing temperature would also allow a larger blocking probe, due to the Ta gap required, in turn generating a larger scanning region for mutations. The invention provides that by modification of the primers to increase the Ta ≥65° C., the specificity of wild-type blocking probes can be increased, and further fold enrichment can be generated compared to previous iterations of the system in the public domain. Moreover, a scanning region of 5-50 bp or more can be achieved and strong enrichment can be generated for both DNA and LNA bases. This further invention is referred to as Highly Optimised Annealing Temperature—Probe Inhibited (HOT_PI) PCR.

HOT_ARMS PCR is recommended for situations where there is priori knowledge of the mutation and HOT PI PCR is recommended where the mutations are unknown and screening needs to take place. These two methods can work separately or in unison. i.e. the methods and other aspects of the HOT ARMS PCR invention and HOT PI PCR invention may be combined. Therefore, all aspects, embodiments and optional features of the first aspect of the invention may apply to the aspect of the invention that requires a blocking probe (HOT PI PCR). HOT_ARMS PCR may also utilise a blocking probe.

In the aspect of the invention requiring a blocking probe (HOT PI PCR) the forward and/or reverse primers may be fully complementary to the corresponding wild type sequence (for example, they may not comprise a mismatching base pair), with exception of the 5′ tag if present. In the aspect of the invention (HOT PI PCR) the 3′ nucleotide of the forward and/or reverse primers may be complementary to the corresponding wild type sequence. For example, the forward and/or reverse primers may not comprise a mismatching base pair at the 3′ nucleotide, unlike the HOT ARMS PCR method).

In another embodiment of the aspect of the invention requiring a blocking probe (HOT PI PCR), the terminal 3′ nucleotide of either the forward or reverse primer is arranged to form a base pair with the mutation of interest in the target nucleic acid having a variant sequence.

The blocking probe may comprise a nucleic acid that is fully complementary to the non-variant sequence and/or non-targeted variant sequence(s). The blocking probe may prevent at least the 3′ end of the forward and/or reverse PCR primers from hybridising to the non-variant sequence and/or non-targeted variant sequence(s), thereby blocking polymerisation in a PCR. The blocking probe may hybridise to the entire region between the forward and reverse primers and/or extend beyond the 5′ end of the forward or reverse primers. The blocking probe may hybridise to the entire region between the forward and reverse primers.

In another embodiment, the blocking probe may hybridise to a region of the wild-type nucleic acid that at least partially overlaps with a region of binding of the forward and/or reverse primer, thereby at least partially blocking hybridisation of the forward and/or reverse primers respectively. In one embodiment, the blocking probe hybridisation region may extend at least 1 bp into the forward and/or reverse primer binding region. In another embodiment, the blocking probe hybridisation region may extend at least 2 bp into the forward and/or reverse primer binding region. In another embodiment, the blocking probe hybridisation region may extend at least 3 bp into the forward and/or reverse primer binding region. In another embodiment, the blocking probe hybridisation region may extend at least 4 bp into the forward and/or reverse primer binding region. In another embodiment, the blocking probe hybridisation region may extend at least 5 bp into the forward and/or reverse primer binding region. In another embodiment, the blocking probe hybridisation region may extend over the whole forward and/or reverse primer binding region. In another embodiment, the blocking probe hybridisation region may extend over the whole forward and/or reverse primer binding region and past the 5′ end. The blocking probe may comprise or consist of the same sequence as the forward or reverse primer, except it is modified to prevent polymerisation therefrom, such that it cannot act as a primer for polymerisation.

The blocking probe may be arranged to hybridise to the anti-sense strand.

The blocking probe may comprise nucleic acid that is not capable of acting as a primer for polymerase. For example, the probe may be 3′ tagged with a molecule that sterically prevents polymerase from docking and carrying out the polymerase chain reaction. In one embodiment, the probe may comprise a 3′ phosphate group, which is chain terminating. In another embodiment, the probe may comprise 3′ chain terminating analogues, such as 3′-dA, 3′-dG, 3′-dC, and 3′-dT, or 3′ minor groove binder (MGD) in order to prevent polymerisation. Additionally or alternatively, the probe may comprise non-complementary bases at the 3′ end of the probe, such that the polymerase can't extend beyond them. Therefore, the blocking probe may comprise any suitable length for hybridisation with the region between primer binding sites or at primer binding sites.

The blocking probe may comprise DNA. In one embodiment, the blocking probe comprises a nucleotide analogue, such as LNA, PNA, or BNA at any position. In one embodiment, the blocking probe comprises a combination of one or more nucleotide analogues, such as LNA, PNA, or BNA, at any position, and DNA. In one embodiment, the blocking probe comprises one or more nucleotide analogues, such as LNA, PNA, or BNA, at the known or predicted site of the variation, such as mutation. Such sites may otherwise be known as “hotspots”. For example, KRAS exon 2 codons 12 and 13 is a known hot spot that contains 99.9% of mutations by frequency in this exon for cancer. Frequencies are readily available to the skilled person, such as on the website COSMIC. Many mutation hot spots exist in cancer and these are good targets for picking up mutations in a large number of patients with unknown mutations beforehand. For example in PIK3CA codon 542 and 545 are hot spots present in 15% of colorectal cancer patients and these could be targeted together by wild-type blocking. For example, 60-90% of melanoma patients depending on the region in the world have BRAF V600E mutations. In one embodiment, the nucleotide analogue is LNA. In one embodiment the blocking probe comprise DNA with between 1 and 20 LNA bases. In another embodiment the blocking probe comprise DNA with between 3 and 15 LNA bases. In another embodiment the blocking probe comprise DNA with between 3 and 6 LNA bases. In another embodiment the blocking probe comprise DNA with 6 LNA bases.

Nucleotide analogues, such as LNA, can help to clamp the blocking probe to the non-variant nucleic acid and/or non-targeted variant nucleic acid.

The blocking probe may be at least about 6 bp in length. The blocking probe may be about 100 bp or less in length. The blocking probe may between about 6 and about 100 bp in length.

The blocking probe may have a Ta below the Ta of the forward and/or reverse primers, such that it is arranged to hybridise to the non-variant nucleic acid and/or non-targeted variant nucleic acid before the forward and/or reverse primers. For example, the blocking probe may have a Ta of at least 8° C. above the Ta of the forward and/or reverse primers. In another embodiment, the blocking probe may have a Ta substantially the same as the forward and/or reverse primers. In another embodiment, the blocking probe may have a Ta of at least 1° C. above the Ta of the forward and/or reverse primers. In another embodiment, the blocking probe may have a Ta of between 1° C. and 20° C. above the Ta of the forward and/or reverse primers.

For detection of target nucleic acid having a KRAS mutation, the blocking probes may comprise or consist of the following sequence 5′ ACTGAATA[T]AAACTTGTGGTAGTTGGAGCT[G][G]T[G][G]CGTAGGCA[A]GAGTG CCTT-PHO 3′ (SEQ ID NO: 37), wherein the nucleotides in brackets represent LNA, or other nucleotide analogue bases; or the complementary sequence thereof. This may be used in combination with a forward primer of the sequence 5′ GGGCCGGCCCTTATAAGGCCTGCTGAAAATGACTGA 3′(SEQ ID NO: 38), and/or reverse primer of the sequence 5′ GGGCCGGCCCTTCTGAATTAGCTGTATCGTCAAGGC 3′(SEQ ID NO: 39).

In another embodiment for the detection of target nucleic acid having a KRAS mutation, the blocking probes may comprise or consist of the following sequence 5′ AAGGCAC[T]CTTGCCTACG[C][C]A[C][C]AGCTCCAACTACCACAAGTTTA[T]ATTC AGT-PHO 3′ (SEQ ID NO: 40), wherein the nucleotides in brackets represent LNA, or other nucleotide analogue bases; or the complementary sequence thereof. This may be used in combination with a forward primer of the sequence 5′ GGGCCGGCCCTTATAAGGCCTGCTGAAAATGACTGA 3′(SEQ ID NO: 41), and/or reverse primer of the sequence 5′ GGGCCGGCCCTTCTGAATTAGCTGTATCGTCAAGGC 3′(SEQ ID NO: 42).

The blocking probe may be provided in an amount of 50 nM and 500 nM. The blocking probe may be provided in an amount of about 100 nM.

HOT PCR

According to another aspect of the present invention, there is provided a method of rapid polymerase chain reaction amplification of a target nucleic acid in a pool of nucleic acid, the method comprising:
providing a forward and reverse PCR primer pair capable of hybridising to the target nucleic acid for PCR amplification of the target nucleic acid, wherein the forward and reverse PCR primers each comprise a 5′ tag of non-complementary nucleotides and have a minimum annealing temperature (Ta) of 65° C.;
carrying out a PCR with one or more first cycle temperature profiles, such that the 5′ tag of non-complementary nucleotides of the forward and reverse PCR primer pair become incorporated into the PCR amplicons,
wherein the first cycle temperature profile provides an annealing temperature of at least 65° C. and which is suitable for the annealing of the forward and reverser primer pair such that they hybridise to the target nucleic acid, and
carrying out a PCR with one or more second cycle temperature profiles in order to amplify the PCR amplicons from the PCR cycle(s) of the first cycle temperature profile,
wherein the second cycle temperature profile provides an annealing temperature that is higher than the first cycle temperature profile and which is suitable for the annealing of the PCR amplicons.

The invention herein recognises the advantage of the increase in free energy generated by raising the annealing temperature to ≥65° C., which would prevent inappropriate base-pairing and perfect base matches would be more stable resulting in increased oligonucleotide specificity. Moreover, the implementation of 5′ tags can increase selectivity for PCR amplicons over DNA template, reducing the amount of non-specific amplification in PCR, especially in exponential phase. When tagged primers initially bind DNA, the tag can only partially bind DNA as it does not contain the tag sequence. When primers form amplicons they are incorporated. Thus, when amplicons are formed, the tag sequence is present in the template for primers to bind with perfect complementarity rather than partial complementarity and this causes further gains in the maximum annealing temperature. This novel finding generates the ability to generate a 2-phase touch-up cycling PCR with phase 1 having a lower maximum potential annealing temperature than phase 2. During phase 2 the raised annealing temperature which is beyond the annealing temperature that can be achieved in phase 1 will provide preferential amplification of amplicons rather than DNA. For example, in cycle 1 as amplicons are forming, the maximum annealing temperature may be 71° C. After cycle 1, the annealing temperature may be increased to 72-80° C. Since polymerase activity is between 68-80° C., extension can still occur. Cycling can alternate between 95° C. and 72-80° C. and this greatly reduces the amount of time spent ramping up and down to standard annealing temperatures of 45-60° C. Moreover, PCR can be carried out using 1 cycling phase PCR at >65° C. and result in increased selectivity whereby tagged primers after cycle 2 can bind amplicons preferentially due to tag incorporation, creating perfect complementarity. When cycling from 95° C. down to an annealing and/or extension temperature the primer can bind with perfect tag sequence complementarity and this means the primer will bind amplicons first before DNA. Dramatic increases in specificity result in less failure of PCR; reduced formation of non-specific products; increased multiplexing capability and increased amplification of areas of the genome containing difficult template which has high similarity with other sequences. This also allows faster amplification of DNA as less time is required cycling to low annealing temperatures; saving time ramping and reducing overall PCR time to create rapid PCR cycling. Such a method may be referred to as “High Optimized Ta-PCR” herein.

The method may be for detection of a target nucleic acid in the pool of nucleic acid. The method may further comprise detecting any PCR product or amplification in real-time, wherein the detection of a PCR product or amplification in real-time confirms the presence of the target nucleic acid in the pool of the nucleic acid.

The PCR cycling profile may have one cycling phase or more. The annealing temperature of the cycling phases may be between 65-90° C., and optionally may differ from each other.

Between 1 and 60 cycles of the first cycle temperature profile may be provided. In another embodiment, between 1 and 15 cycles of the first cycle temperature profile may be provided. In another embodiment, between 1 and 10 cycles of the first cycle temperature profile may be provided. In another embodiment, between 5 and 15 cycles of the first cycle temperature profile may be provided. In another embodiment, between 1 and 60 cycles of the first cycle temperature profile may be provided. In another embodiment, about 10 cycles of the first cycle temperature profile may be provided.

The melt temperature phase of the first cycle temperature profile may be between 1 and 60 seconds. Additionally or alternatively, the annealing temperature phase of the first cycle temperature profile may be between 1 and 60 seconds.

The melt temperature phase of the first cycle temperature profile may be between 1 and 60 seconds. Additionally or alternatively, the annealing temperature phase of the first cycle temperature profile may be between 1 and 60 seconds.

The melt temperature phase of the first cycle temperature profile may be about 1 second. Additionally or alternatively, the annealing temperature phase of the first cycle temperature profile may be between 1 and 10 seconds.

Between 1 and 60 cycles of the second cycle temperature profile onwards may be provided. In another embodiment, between 1 and 40 cycles of the second cycle temperature profile may be provided. In another embodiment, between 1 and 30 cycles of the second cycle temperature profile may be provided. In another embodiment, between 1 and 20 cycles of the second cycle temperature profile may be provided. In another embodiment, between 5 and 40 cycles of the second cycle temperature profile may be provided. In another embodiment, between 30 and 40 cycles of the second cycle temperature profile may be provided. In another embodiment, about 30 cycles of the second cycle temperature profile may be provided. In another embodiment, about 40 cycles of the second cycle temperature profile may be provided.

The melt temperature phase of the second cycle temperature profile onwards may be between 1 and 60 seconds. Additionally or alternatively, the annealing temperature phase of the second cycle temperature profile may be between 1 and 60 seconds.

The melt temperature phase of the second cycle temperature profile onwards may be between 1 and 10 seconds. Additionally or alternatively, the annealing temperature phase of the second cycle temperature profile may be between 1 and 10 seconds.

The melt temperature phase of the second cycle temperature profile onwards may be about 1 second. Additionally or alternatively, the annealing temperature phase of the second cycle temperature profile may be between 1 and 10 seconds.

The melt temperature phase of the second cycle temperature profile may be about 1 second. Additionally or alternatively, the annealing temperature phase of the second cycle temperature profile may be between 1 and 5 seconds.

The PCR may comprise the following temperature profiles (≥95° C. for 1-20 min)×1, (≥90° C. for 1-60 sec, then ≥65° C. for 1-60 sec)×1-60.

The PCR may comprise the following temperature profiles (≥95° C. for 1-20 min)×1, (≥90° C. for 1-60 sec, then ≥65° C. for 1-60 sec, then ≥68° C. for 1-60)×1-60. The skilled person will recognise that the timings may be adjusted provided that sufficient amplification occurs. The PCR may comprise the following temperature profiles (≥95° C. for 1-20 min)×1, (≥90° C. for 1-60 sec, then ≥65° C. for 1-60 sec)×1-60, (≥68° C. 30 sec-10 min)×1.

The PCR may comprise the following temperature profiles (≥95° C. for 1-20 min)×1, (≥90° C. for 1-60 sec, then ≥65° C. for 1-60 sec, then ≥68° C. for 1-60)×1-60, (≥68° C. 30 sec-10 min)×1.

The PCR including the first and second cycle temperature profiles may comprise the following temperature profiles (≥95° C. for 1-20 min)×1, (≥90° C. for 1-60 sec, then ≥65° C. for 1-60 sec)×1-60, and (≥90° C. for 1-60 sec, then ≥65° C. for 1-60 sec)×1-60.

The PCR including the first and second cycle temperature profiles may comprise the following temperature profiles (≥95° C. for 1-20 min)×1, (≥90° C. for 1-60 sec, then ≥65° C. for 1-60 sec)×1-60, and (≥90° C. for 1-60 sec, then ≥65° C. for 1-60 sec)×1-60, (≥68° C. 30 sec-10 min)×1.

The skilled person will recognise that the above timings may be adjusted provided that sufficient amplification occurs.

The method of highly specific polymerase chain reaction amplification may be used with and for all other aspects of the present invention as appropriate. For example the method of highly specific polymerase chain reaction amplification may be used in combination with the method herein requiring a blocking probe and/or with the method of the first aspect of the invention herein for the detection of target nucleic acid having a variant sequence. In particular, the method of the invention for all aspects of the invention require a PCR amplification step, and such PCR amplification may use the method of highly specific polymerase chain reaction amplification according to the invention.

The methods herein may be for detecting and/or amplifying a single target nucleic acid species, or may be for detecting and/or amplifying a plurality (i.e. two or more) of different target nucleic acid species, for example in a single reaction/reagent.

The skilled person will understand that standard additional components, for example polymerase, buffer and dNTPs, may be provided in any of the methods, compositions and kits provided herein in order to carry out a PCR reaction.

### Definitions

The terms “variant” or “variant sequence” when used in the context of a nucleic acid sequence may compromise a mutation or variation of a sequence relative to a wild-type sequence. In one embodiment, a variant is a mutant and a variation is a mutation. The term “variant” or “variant sequence” when used in the context of a nucleic acid sequence may also be used to refer to a nucleic acid that is different in sequence to a more common and/or normal nucleic acid that may be present in a pool of nucleic acids. Moreover, wild-type and/or normal sequence may be known as the sequence which is considered normal and/or common for the pool nucleic acids. Therefore, a variant may comprise a nucleic acid sequence that is different in sequence relative to the sequence of another nucleic acid, which may be termed “non-variant” or “wild-type”, in the pool of nucleic acids.

Mutation refers to a change in the nucleic acid sequence such that a novel sequence is generated which is different from the wild-type sequence (i.e. the original sequence which does not contain a mutation). Mutations include (i) Single Nucleotide Variant (SNV) which is a change in a single nucleotide of sequence, (ii) DNA rearrangement and DNA deletion, where a novel sequence is created by juxtaposition of pre-existing sequences and (iii) as DNA insertion and DNA amplification whereby insertion of extra sequences adjacent to pre-existing sequence results in generation of a novel sequence.

The terms “forward primer” and “reverse primer” are used herein to refer to the primer pair necessary for PCR. One of the primers will contain the mutation-specific 3′ base which enable the specific amplification of mutation-containing target nucleic acid whilst target nucleic acid containing wild-type sequence is not amplified. The skilled person will understand that one of these pair will anneal to the sense strand of a target sequence and the other will anneal to the anti-sense/complementary strand. Typically the skilled person will understand that the design is flexible such that the 3′ base which is specific for the mutation may be in either the forward or reverse primer

Reference to a “non-complementary nucleotides” in the context of the 5′ tag is understood to mean that the 5′ tag sequences is generally non-complementary to the target sequence as a whole. However, this does not preclude that one or two of the nucleotides of the 5′ tag may align with, and be complementary to, a nucleotide of the target nucleic acid.

Tm (melting temperature) is understood to be the temperature at which 50% of the DNA is melted (i.e. the strands are separated). The amount of strand separation, or melting, can be measured by the absorbance of the DNA solution at 260 nm.

References to sequence identity may be determined by BLAST sequence alignment (www.ncbi.nlm.nih.gov/BLAST/) using standard/default parameters or MFEprimer (http://mfeprimer.igenetech.com/). For example, the sequence may have at least 99% identity and still function according to the invention. In other embodiments, the sequence may have at least 98% identity and still function according to the invention. In another embodiment, the sequence may have at least 95% identity and still function according to the invention.

The skilled person will understand that optional features of one embodiment or aspect of the invention may be applicable, where appropriate, to other embodiments or aspects of the invention.

Embodiments of the invention will now be described in more detail, by way of example only, with reference to the accompanying drawings.

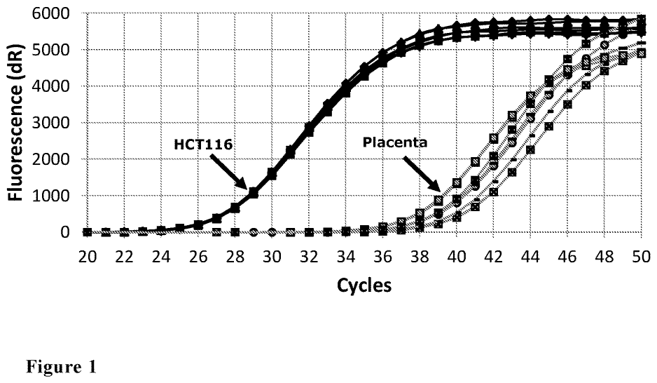

FIG. 1—HOT_ARMS PCR

This is a comparison of HOT_ARMS 1 primers tested in the cell line HCT116 (50% MAF) and the placental DNA showing wide separation. Eight replicates are shown for each demonstrating the precision of the test.

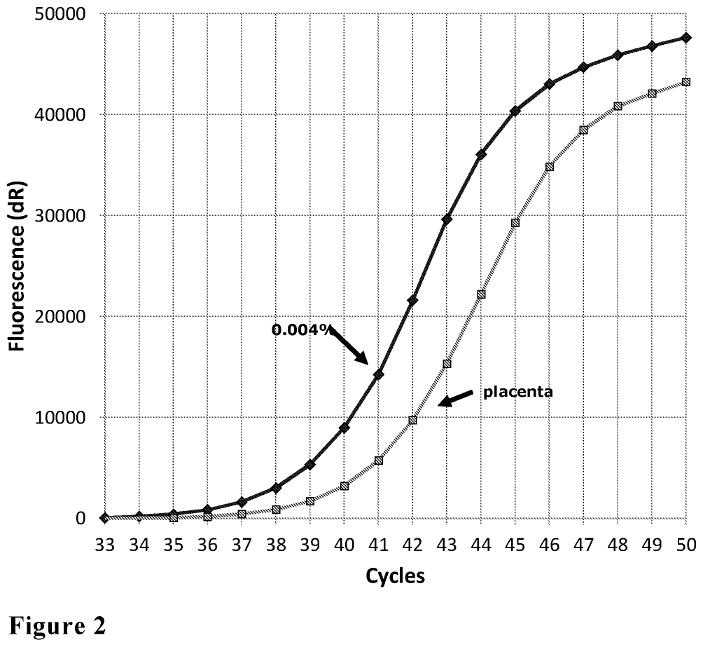

FIG. 2—The limit of detection of HOT_ARMS PCR.

HOT_ARMS1 was tested to its limit. Cell line DNA was spiked into placenta DNA. Mutant alleles at a frequency of 0.004% could be detected and discriminated from pure placental DNA.

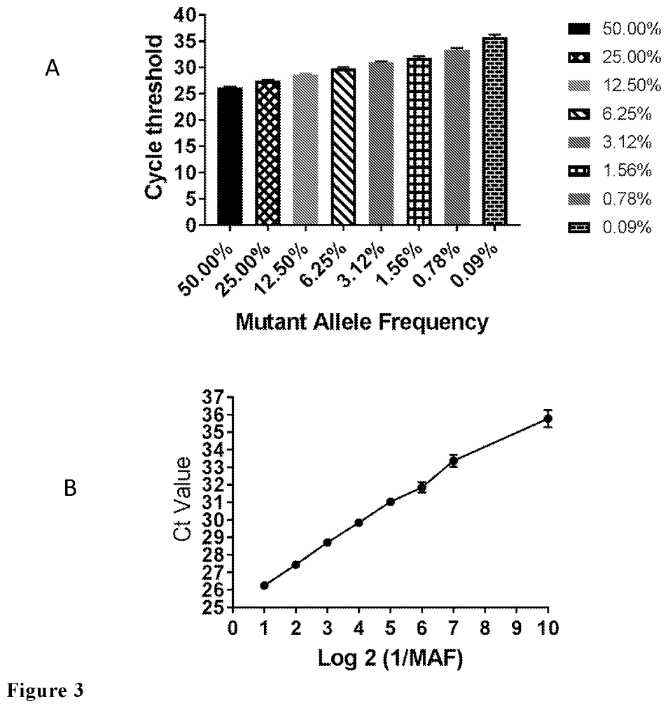

FIG. 3—Dynamic range and efficiency of HOT_ARMS PCR

The dynamic range and efficiency of HOT_ARMS1 was tested on template with mutant allele frequency (MAF) ranging from 50%-0.09%. FIG. 3A is a graphical representation of the Ct values (mean+1 standard deviation of 8 replicates) over this range. FIG. 3B is a plot of mean Ct (+1 standard deviation) against log 2 (1/MAF). The slope of the curve indicates an efficiency of 105% indicating that this could possibly be used for quantification of mutant alleles.

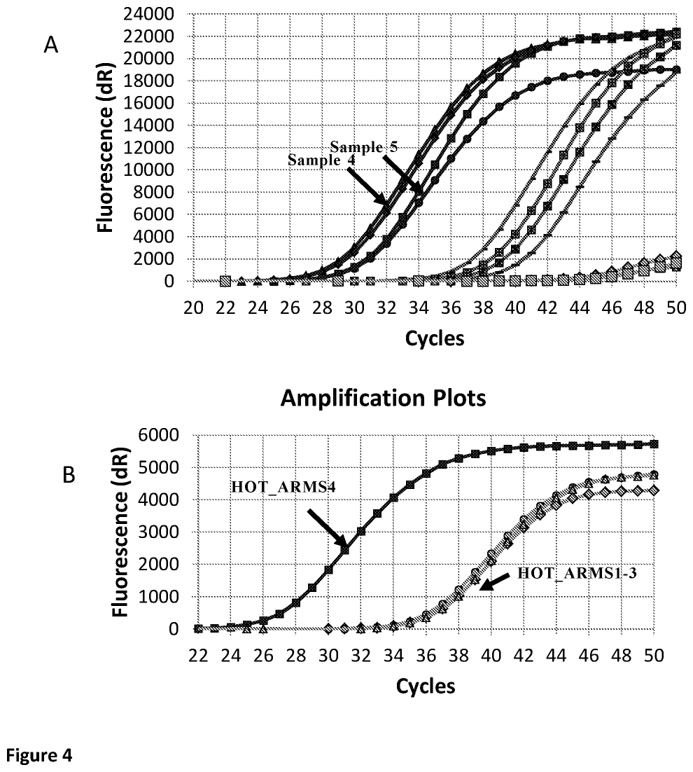

FIG. 4—The utility of HOT_ARMS PCR on DNA from formalin-fixed tissue

HOT_ARMS PCR works on DNA derived from formalin fixed tissue. Several samples which had been previously genotyped were tested. FIG. 4A shows the results of 10 samples tested with HOT_ARMS2; samples 4 and 5 are clearly positive whilst the remaining samples are negative. All samples also underwent blind genotyping. FIG. 4B shows the results of sample 14 with HOT_ARMS 1-4 run as a panel. It is positive for HOT_ARMS 4 but negative for the others.

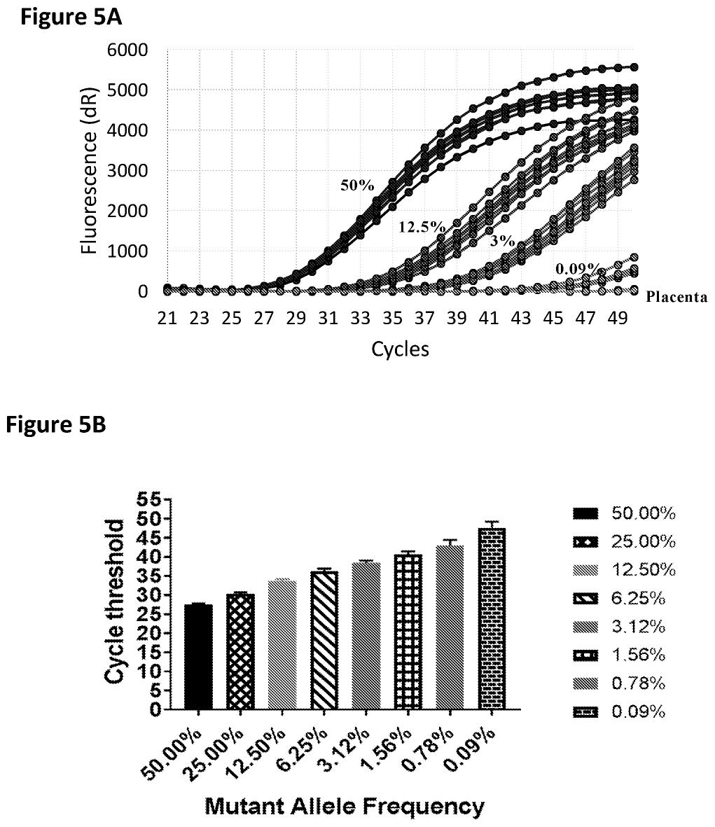

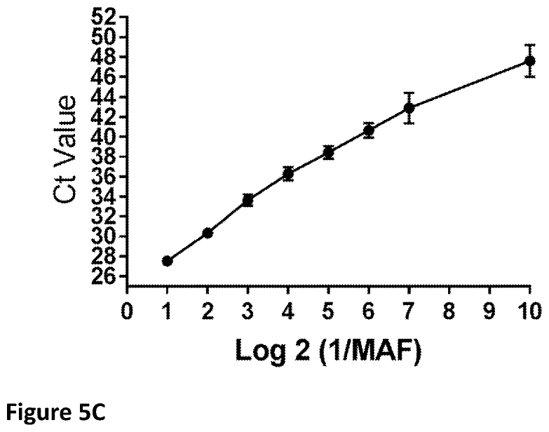

FIG. 5—HOT_ARMS PCR with combined GC tag and 3′LNA

In attempt to improve the HOT_ARMS PCR assay, the HOT_ARMS1 primers were modified to include a locked nucleic acid (LNA) at the 3′ base. FIG. 5A shows that this resulted in complete abolition of non-specific priming of the placental DNA but at the cost of reduced efficiency. The Ct values shown are 20 cycles less than the actual value and have been amended for the purposes of the graphical representation. The Ct values for the cell line at different % MAF came up much later than the equivalent template was tested with GC-tagged primers alone. The dynamic range and efficiency was tested on template with mutant allele frequency (MAF) ranging from 50%-0.09%. FIG. 5B is a graphical representation of the Ct values (mean+1 standard deviation of 8 replicates) over this range. FIG. 5C is a plot of mean Ct (+1 standard deviation) against log 2 (1/MAF). The slope of the curve indicates an efficiency of 113% indicating that whilst giving a clearer “yes/no” answer, this could not be used for quantification of mutant alleles.

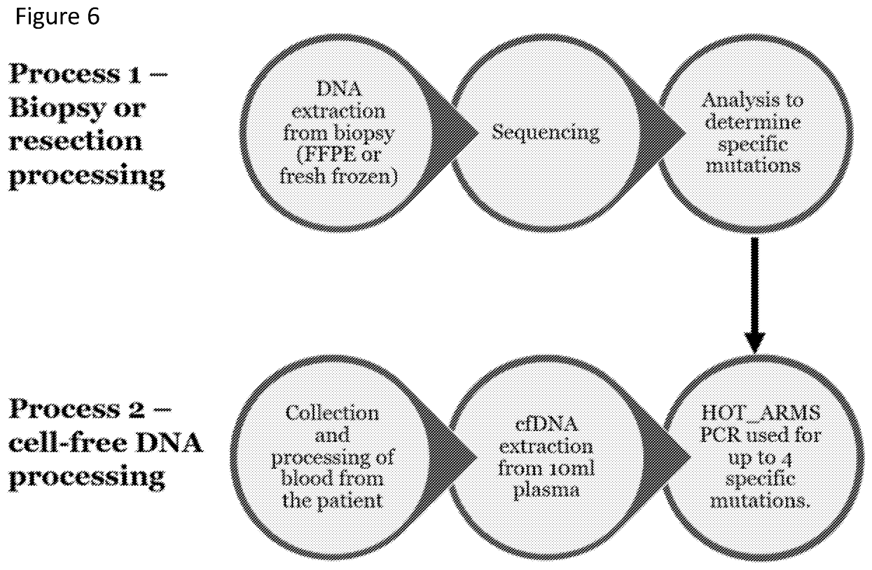

FIG. 6. The process for tumour surveillance by HOT_ARMS PCR.

Process 1 outlines the pathway for tumour specimen mutation detection. Whole genome sequencing will allow the maximum probability for identifying tumour mutations in cell-free DNA. However, other mutation detection methodologies can be used for smaller scale panel systems. Process 2 represents the pathway for cell-free DNA mutation detection. The cell-free DNA will be divided up in order to detect up to 4 or more specific mutations. Lower total DNA input which is distributed over a number of targets will allow greater sensitivity to be achieved via reduced wild-type bleed through as shown in table 6. Moreover, a panel of 4 or more mutations will increase the chances for mutation discovery. Mutation discovery probabilities are based on the heterogeneity of tumour mutations and location of the tumour. The greater the number of targets the increased likelihood for true mutation detection. This is especially important with most cancer treatments which can drive tumour homogeneity and minor clone mutation signals can be lost. Both processes can be carried out individually or simultaneously apart from the last step in process 2 which requires tumour specimen mutation identification beforehand.

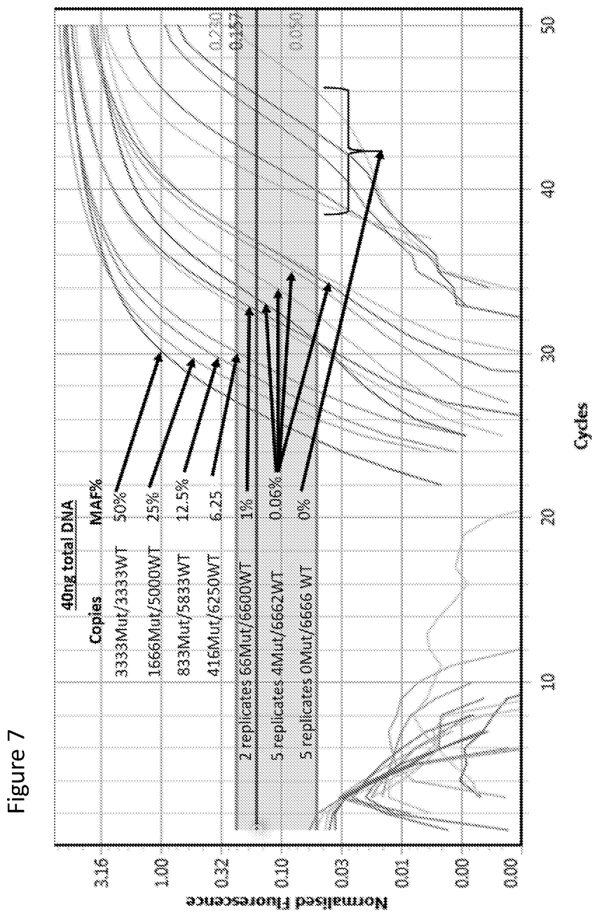

FIG. 7. Amplification plot for HOT_ARMS 12 (BRAF V600E) rapid testing.

HOT_ARMS assays with Act >15 (40 ng total DNA) between 50% MAF and wild-type can undergo rapid 30-minute testing with clear 0.06% (4 mutant copy) detection when utilising a fast cycling mastermix and thermocycler. Rapid testing is made possible by the extremely high specificity HOT_ARMS primers achieve, as shown in table 8. HOT_ARMS assays with lower specificity; Act <15 between 50% MAF and wild-type will obtain lower sensitivities of 0.5% MAF (33 mutant copies). However, with the addition of 3′ LNA, the specificity can be increased and 0.1-0.2% MAF can be detected. The magnetic induced cycler (MIC) PCR machine was utilised to reduce the total PCR time (50 cycles) to 30 minutes including melt-curve.

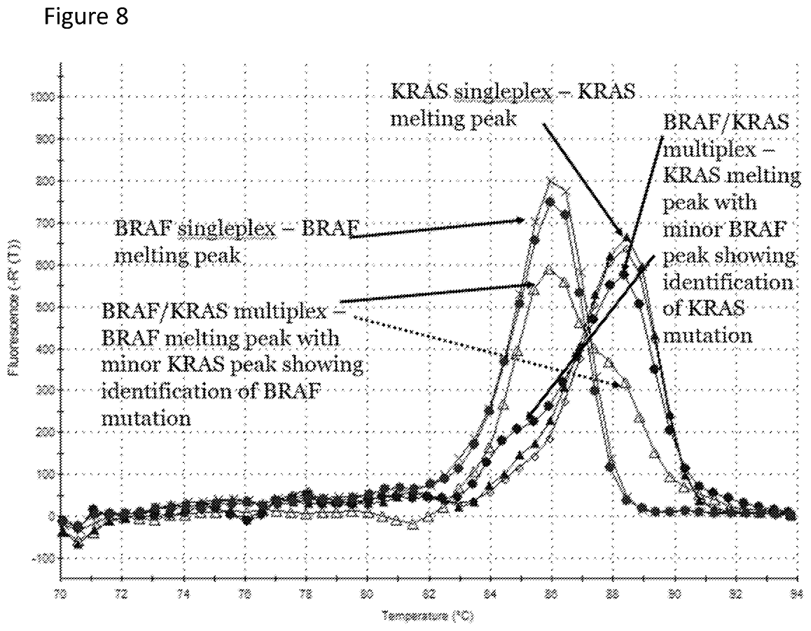

FIG. 8. Melting peak analysis for HOT_ARMS 12 (BRAF V600E) and HOT_ARMS 1 (KRAS G13D) multiplexing.

Ct values are demonstrated in table 9 where 0.06% (4 copies) can be detected. Whilst ct values give yes/no answers for mutations; amplicons can be designed to be of different length, resulting in different melting temperatures. Thus, the mutation call can be determined by ct value and its identity by melting peak analysis. Double-stranded DNA binding dyes are utilised here and no probes. HOT_ARMS 1 and 12 melting peaks are shown in duplicate with clean specific peaks. HOT_ARMS 1 and 12 multiplexing demonstrates that some bleed through occurs. However, the bleed through is minimal and allows for the dominant mutant amplicon to be identified; representing the high specificity achieved by HOT_ARMS PCR.

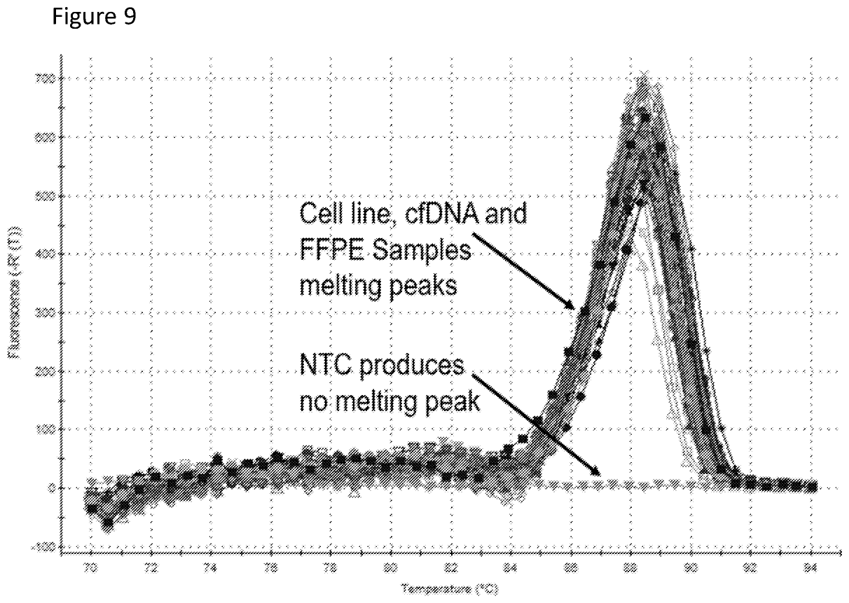

FIG. 9. Clean and specific melting peaks for low DNA input amplification of cfDNA, FFPE DNA and cell line DNA with flat no template control (NTC).

In all instances, 62.5 μg (˜10 copies) can be amplified. Ct values shown in table 10.

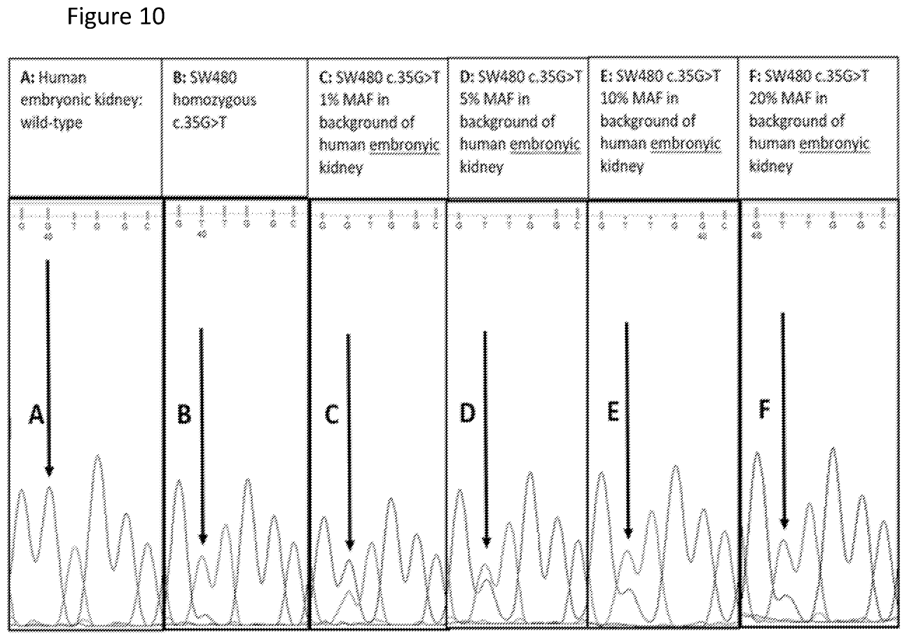

FIG. 10. Sanger sequencing of a 95 base pair amplicon for KRAS exon 2 (codon 12 and 13 shown) which has undergone mutation enrichment by the highly optimised annealing temperature probe inhibited PCR system.

HOT_PI PCR products can undergo mutation detection via sequencing, high-resolution melting analysis, mutation specific probes or digital-droplet PCR. This figure demonstrates the enrichment potential using 100 nM of wild-type blocking probe which allows wild-type to partially amplify for sequencing. Further enrichment can be permitted as shown in table 12. Image A represents wild-type sequence from the HEK293T cell line; Image B shows a homozygous G>T mutation found in the colon cancer cell line SW480 (c.35G>T) on the second DNA base in the image; Image C shows SW480 spiked into HEK293T (wild-type) at 1% mutant allele frequency (MAF); Image D shows SW480 spiked into HEK293T (wild-type) at 5% MAF; Image E shows SW480 spiked into HEK293T (wild-type) at 10% MAF; Image F shows SW480 spiked into HEK293T (wild-type) at 20% MAF. As the limit of detection for Sanger sequencing is 10-20%, image C (1% MAF), D (5% MAF) and E (10% MAF) should be completely wild and image F (20% MAF) should show a small peak for Thymine. However, the mutation enrichment is so strong that the 1% MAF shown in image C becomes easily detectable and Thymine becomes the dominant peak in image D (5% MAF), E (10% MAF) and F (20% MAF). This figure is purely for demonstrations of enrichment potential rather than its true limit of sensitivity and fold-enrichment which can be obtained by pyrosequencing, deep-sequencing or mutation specific probes with higher blocker concentrations.

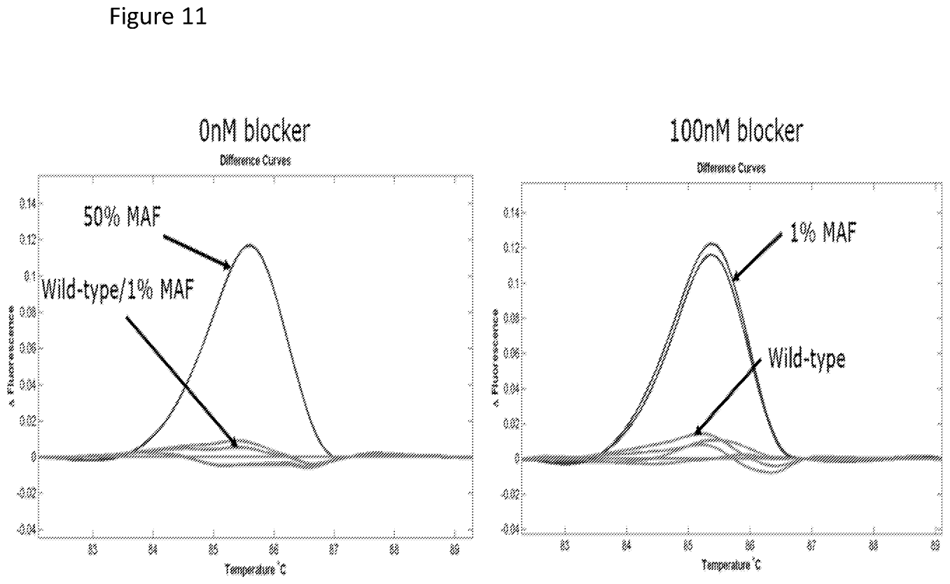

FIG. 11. HOT_PI PCR mutation enrichment demonstrated by high-resolution melting analysis difference curves.

Without wild-type blocking probe; 1% MAF does not differ from wild-type DNA. However, with 100 nM wild-type blocking probe, 1% MAF shows a large difference in melting behaviour representing a mutation. 0.1% MAF (8 mutant copies) can be detected using high resolution melting analysis with 100 nM wild-type blocking probe. HOT_PI PCR products can undergo mutation detection via sequencing, high-resolution melting analysis, mutation specific probes or digital-droplet PCR. This figure demonstrates the enrichment potential using 100 nM of wild-type blocking probe which allows wild-type to partially amplify for high-resolution melting analysis. Further enrichment can be permitted as shown in table 12. High-resolution melting analysis depends on heteroduplex formation. Thus, homozygous mutations can only be detected by ct value rather than melting behaviour. Greater mutation enrichment can be achieved with higher concentrations of blocking probe but heteroduplex formation restricts the method as low MAF samples begin forming mutant homoduplexes which disrupt the analysis. This figure is purely for demonstrations of enrichment potential rather than its true limit of sensitivity and fold-enrichment which can be obtained by pyrosequencing, deep-sequencing or mutation specific probes with higher blocker concentrations.

### EXAMPLE 1—HOT_ARMS PCR: A SIMPLE AND EXQUISITELY SENSITIVE METHOD FOR MUTATION DETECTION

Summary

Knowledge of tumour mutations underpins precision medicine in the management of cancer patients. This knowledge can also support other care pathways such as tumour surveillance. Not infrequently, however, the proportion of mutant alleles in patient-derived DNA samples is very low and the presence of contaminating wild-type DNA may make mutation detection unreliable. In order to circumvent this problem, the High Optimised Ta Amplification Refractory Mutation System (HOT_ARMS) PCR has been developed in accordance with the invention herein. This depends on modification of allele-specific primers (through addition of a GC-rich tag and/or incorporation of modified nucleic acids) to enable PCR to be performed at an annealing temperature ≥65° C. HOT_ARMS PCR does not require special probes and can be performed on standard real-time PCR machines without the need for expensive equipment.

13 different mutations were tested (including single nucleotide variants and insertion-deletion mutations) in template derived from cell lines and formalin-fixed tissue. Robust mutation detection was shown with a limit-of-detection as low as 0.004% mutant allele frequency (MAF). The assay has a wide dynamic range and excellent precision even at low MAF. Each target tested using HOT_ARMS PCR required little or no optimisation and the tests could be multiplexed. Blind testing of 10 FFPE cases and 11 circulating tumour DNA cases with known KRAS mutation correctly identified the genotype of each case thus confirming the accuracy of the assay.

In summary, HOT_ARMS PCR is an extremely simple, robust and sensitive test. It is a single stage closed-tube test which could transform cancer patient management by widening access to genetic testing. The speed of the test means it can be established in the hospital out-patient and even the primary care setting.

Materials and Methods

DNA Extraction

DNA from cell lines (see Table 14 for list of cell lines used) was extracted using the GenElute mammalian genomic DNA miniprep kit (Sigma-Aldrich, U.S.A) using the manufacturers protocol.

DNA was extracted from formalin-fixed paraffin-embedded (FFPE) from 10 cases of colorectal cancer which had previously been tested for KRAS mutation. Sections were reviewed to confirm that, semi-quantitatively, tumour cells comprised at least 30% of the total cellular population. Twenty μM section curls were cut from tumour blocks, dewaxed in xylene and digested completely at 55° C. with proteinase K (Qiagen) in accordance with the QIAamp FFPE DNA extraction protocol. DNA was extracted using the Qiamp FFPE DNA extraction kit (Qiagen) in accordance with manufacturer's instructions.

DNA Dilutions and Limit of Detection

Placental DNA and some of the mutation-containing cell line DNA was purchased from commercial sources. All DNA was quantified using a Nanodrop spectrophotometer 2000c (Thermo Scientific) and 260:280 absorbance ratio of 1.8-2.0 was taken as indicative of good quality. DNA was diluted to a final concentration of 20 ng/μl with nuclease free water (Qiagen, Germany). DNA from cell lines with known mutations was spiked into placental DNA containing wild type sequence. Templates samples were prepared containing mutant alleles frequencies (MAF) ranging from 50% down to 0.004%.

Primer Design and Modification

(a) Primer Design

Two types of mutation were tested i.e. Single Nucleotide Variants (SNV) and insertion-deletion (Indel) mutations. In both cases, a novel sequence is created—for SNVs this is due to the substitution of a wild-type base for a mutant base. For Indels, the insertion or deletion of bases results in the disruption of the wild type sequence. In both cases, primers were designed to produce short products (60-80 bp in some tests and 50-110 bp in other tests) and one of the primers contained a mutation specific base at the 3′ end of the sequence. The other primer contained wild-type sequence. In total, 13 different mutations were tested in 8 different codons in 5 different genes (see Table 1). The mutations tested in BRAF, EGFR, KRAS and PIK3CA were SNV whilst the mutation detected in APC was a frameshift deletion mutation.

HOT_ARMS PCR works on the principle that a high Ta will improve the specificity of the PCR. As a rule of thumb, the Ta is usually 5° C. lower that the melting temperature (Tm). The definition of Tm is the temperature at which 50% of the DNA is melted (i.e. the strands are separated) and this is the physical property of DNA which is used by primers design software packages. Primers were initially designed in Primer 3 [15] mostly according to the standard rules [16] and then modifications were made to raise the Tm/Ta.

In this non-limiting example HOT_ARMS primers were designed as follows: (i) minimum primer length 20 bases, optimum primer length 25 bases and maximum primer length 30 bases (length/bases does not include 5′ tag); (ii) minimum GC content 30%, optimum GC content 45%, maximum GC content 60% (GC content does not include 5′ tag content); (iii) minimum Tm (melting temperature) 60° C., optimum Tm 65° C., maximum Tm 85° C.; (iv) minimum amplicon length 50 or 60 bp, maximum amplicon length 110 bp; (v) primer dimer free energy (ΔG)<−6 and Tm<60° C.; (vi) hairpin ΔG<−6 and Tm<60° C.; (vii) 3′ base is specific for the mutation in either the forward or reverse primer; (viii) Maximum Tm difference between forward and reverse primer 3° C.

Examples of how the Mutation Specific Primers are Designed for Different Mutation Scenarios:

Hypothetical wild-type sequence of 30 nucleotides is mutated in different ways to model the different possibilities which would occur in practice. Mutations are highlighted and both the original and the new mutated sequence are shown. The ARMS specific primer which would work only on the mutant sequence are shown.

Hypothetical sequence:
GATCGGATTGGCAAATTAGCCGTAGGCCGG

Example SNV: Nucleotide Highlighted in Bold Changes from T to C

Original sequence:
GATCGGATTGGCAAATTAGCCGTAGGCCGG
New sequence:
GATCGGATCGGCAAATTAGCCGTAGGCCGG
Primer sequence:
GATCGGATC

Example Deletion: Nucleotide(s) Highlighted in Bold Deleted

Original sequence:
GATCGGATTGGCAAATTAGCCGTAGGCCGG
New sequence:
GATCGGATGGCAAATTAGCCGTAGGCCGG
Primer sequence:
GATCGGATG
Original sequence:
GATCGGATTGGCAAATTAGCCGTAGGCCGG
New sequence:
GATCGGATAGCCGTAGGCCGG
Primer sequence:
GATCGGATA

Example Insertion: Nucleotide Highlighted in Bold is Inserted

Original sequence:
GATCGGATTGGCAAATTAGCCGTAGGCCGG
New sequence:
GATCGGATTTGGCAAATTAGCCGTAGGCCGG
Primer sequence:
GATCGGATTT

Example Amplification: Nucleotides Highlighted in bold amplified ×4

Original sequence:
GATCGGATTGGCAAATTAGCCGTAGGCCGG
New sequence:
GATCGGATTGGCTGGCTGGCTGGCTGGCAAATTAGCCGTAGGCCGG
Primer sequence:
GATCGGATTGGCT

Example Rearrangement: Nucleotides Highlighted Bold are Moved to Another Site in the Sequence in Bold

Original sequence:
GATCGGATTGGCAAATTAGCCGTAGGCCGG
New sequence:
GATCGGATTGAGGCCGGCAAATTAGCCGTG
Primer sequence:
GATCGGATTGA

(b) Primer Modification

For HOT_ARMS PCR, the primers need to be further modified to increase the Tm. There are two main approaches available i.e. addition of a sequence tag to the primers or incorporation of modified bases into the primers.

(i) Addition of a tag. Extending the length of a primer by adding extra sequences in the form of a 5′ tag will raise the Tm of the primer. The greatest increase will be achieved if the tag contains a high proportion of Guanine and Cytosine bases. Two tags were tested: a 10 base tag with the sequence 5′-gggccggccc-3′ (SEQ ID NO: 35) (predicted to raise the Tm by approximately 20° C.) and a 15 base tag with the sequence with the sequence 5′-gggccgggccggccc-3′ (SEQ ID NO: 36) (predicted to raise the Tm by approximately 25° C.). The tags were added to both the forward and reverse primers.

(ii) Incorporation of modified nucleic acids. Recently developed synthetic nucleic acids (such as Locked Nucleic Acids (LNA), Bridged Nucleic Acids (BNA) and Peptide Nucleic Acids (PNA)) have a much greater binding affinity for the paired base on the opposite DNA strand than naturally occurring nucleic acids [18-20]. Incorporation of synthetic nucleic acids into primers/probes has been used to increase the specificity [18, 21, 22] although the high binding affinity stabilizes double stranded DNA thereby increasing the Tm (by approximately 2° C. per nucleotide). LNA's were tested here (BNA/PNA are predicted to have the same effect). Primers were designed in accordance with the rules above and 3-5 LNAs were included at various sites within the primer in accordance with published recommendations [18, 21-23].

(iii) Combination of tag and LNA. Primers modifications of varying tags and LNAs were tested individually. Based on the results, a new modification of combining tag with LNA was developed. This consisted of the 10 base tag (5′-gggccggccc-3′ (SEQ ID NO: 35)) together with an LNA incorporated as the 3′ base of the mutation specific primer.

HOT_ARMS PCR Protocol and Optimisation

All reactions were undertaken in 0.2 ml tubes with caps (Agilent, U.S.A). Each reaction was performed in a final volume of 10 μl which contained the following components: 2× HotShot Diamond mastermix (Clent Life Science, U.K) which includes a final concentration of 6 mM MgCl₂ and 400 μM dNTPs with stabiliser; EvaGreen dye 20× in water (Biotium, U.S.A); adjusted DNA template; 375 nM each primer and Nuclease-Free water (Qiagen, Germany). Standard nucleic acid primers were purchased from Eurofins (Luxembourg) and LNA containing primers were purchased from Eurogentec, (Belgium).

Primers were designed to have a minimum Tm of 70° C. to allow a Ta ≥65° C. to be used. All primers worked at the same temperature as they were designed that way for minimal optimisation. The initial optimisation used template containing 50% mutant alleles and a single PCR product was confirmed by high resolution melting analysis on a Lightscanner (Biofire Defence, U.S.A). In order to produce a standard protocol which would work for multiple targets, generally a Ta of 71° C. was used. The effect of primer concentration on test performance at low levels of mutant allele frequency (0.125% and 0.0625%) was tested using differing primer concentrations (250 nM-600 nM) with a Ta of 71° C.

Tests were performed on the Stratagene MX3005P real-time machine. The threshold for detection was set using the machine's default parameters (10 standard deviations away from the mean of the baseline fluorescence). For tagged primers, template containing different mutant allele frequencies was tested using the following cycling parameters: (95° C./5 min)×1/(95° C./20 sec; Ta/20 sec; 72° C./20 sec)×50. LNA primers enhance specificity at the expense of PCR efficiency and to test these primers, a modified “touch-up” protocol was used as follows: (95° C./5 min)×1/(95° C./30 sec; 60° C./20 sec; 72° C./20 sec)×20 (95° C./20 sec; Ta/20 sec; 72° C./20 sec)×30. This protocol was also used for the combined tag/LNA primers. A standard protocol can be used with mastermixes or nested PCR to improve efficiency.

HOT_ARMS PCR Assay Testing and Statistical Analysis

Limit-of-detection tests were performed on templates containing varying proportions of mutant allele and compared against placental DNA (containing only wild type DNA). In general, 20 ng of template was used although, at the lower mutant allele frequencies (MAF), the chances of a mutant allele being present follows a Poisson distribution. For this reason, the amount was DNA was adjusted to ensure that there were at least four copies of mutant allele theoretically calculated to be present. This means, by Poisson statistics, at least one copy would always be present. When testing the performance of HOT_ARMS PCR on DNA derived from FFPE tissue, 20 ng of tumour DNA was used.

Short term precision and PCR dynamic range was tested on a series of samples containing doubling dilution spiked-in MAF ranging from 50%-0.09%. Short term precision was tested by repeating the same assay 8 times in a single run and calculating the coefficient of variation of the Ct values. The short term precision was tested at several different mutant allele frequencies. The PCR efficiency for mutant alleles was calculated by plotting the Ct values against the log 2 [1/MAF] and calculating the slope of the curve. A slope value of 1.0 would be indicative of 100% efficiency.

For multiplex analysis, multiple different primers targeting mutations in codon 12 of KRAS and different exons in KRAS/BRAF were tested in a single reaction. Tests were performed on cell line DNA templates containing the different mutations and on placental wild-type template.

Results

HOT_ARMS PCR Robustly Distinguishes Mutant DNA Cell Line from Placental DNA

Primers were designed for 13 different mutations (Table 1) and for the purposes of simplicity the primer pairs for these mutations are referred to a HOT_ARMS1 through to HOT_ARMS13. Gradient PCR showed that HOT_ARMS1 primers produced a single PCR product at 71° C. and so this was adopted as the Ta for all subsequent reactions without further optimisation. Similarly, optimisation of the primer concentration showed that a concentration of 375 nM per reaction was the best across all mutant allele concentration and this was therefore chosen as the final consensus primer concentration.

Firstly the ability to detect mutations in cell line DNA was tested and compared against the non-specific background amplification of placental DNA (FIG. 1, Table 2). Using the normal rules and nomenclature of real-time PCR, detection was defined by the Ct value. For each test, replicates were performed and the mean Ct value of the replicates was used for analysis. Initially primers tagged with a 10 base GC tag were tested. Tests were performed at least in duplicate and all 13 primers showed detection of the cell line DNA (containing 50% mutant alleles) with a mean Ct value around 26 cycles. There was no difference in assay performance whether the underlying mutation was an SNV or an indel mutation.

It is expected that non-specific “bleed-through” amplification will occur in wild-type DNA even with mutation-specific primers and non-specific amplification in placental DNA. Every HOT_ARMS primer pair showed a difference in Ct (ΔCt value) between the cell lines and placenta of at least 10 cycles with a mean ΔCt value of 13. The HOT_ARMS primers were compared with the same primer sequences without the GC tags. When PCR was performed at a Ta of 55° C., the HOT_ARMS primers performed more poorly than the non-tagged primers with a ΔCt value between the cell lines and placenta of 3 and 4 respectively (data not shown). When PCR was performed at a Ta of 71° C., the non-tagged primers did not produce any amplification.

Given the specificity of the HOT_ARMS primers, we tested whether they could be multiplexed. For testing KRAS codon 12/13 mutations, many of the HOT_ARMS primers have a common reverse primer and a mutation specific forward primer (Table 1). This allowed an assay to be set up with several forward primers and a single reverse primer. Both wild-type and mutant templates were tested and it was possible to combine up to 4 forward primers with little change in the performance of the assay i.e. each of the mutations was detected at the expected Ct and the large numbers of primers did not interact to generate false positives when tested on the placental DNA.

Primers tagged with a 10 base GC tag were found to have a higher efficiency than primers tagged with 15 base GC tags.

HOT_ARMS PCR has a Very Low Limit of Detection

DNA from cell lines containing known heterozygous mutations was spiked into placental DNA (Table 6) to produce mutant allele frequencies (MAF) ranging from 50%-0.004%. Limit of detection tests were performed in duplicate and a test was called positive if there was a ΔCt value between test sample and placental DNA of ≥2. The limit of detection was variable ranging from 0.06% MAF for the least efficient primer pairs, down to 0.004% MAF for HOT_ARMS1, the most efficient primer pair (FIG. 2, Table 2). The remaining 12 primer pairs were able to discriminate down to between 0.06% and 0.01% MAF and were not tested further as this was felt to be a sufficient limit of detection.

HOT_ARMS PCR has a Wide Dynamic Range and Excellent Precision

All primer pairs were found to show a very low limit of detection when testing for specific mutations. In order to test the robustness of the assay, the short term precision (also known as the intra-assay variability) was evaluated, which is best evaluated through measuring the coefficient of variation.

Eight replicates were performed for the HOT_ARMS1 primer pair with spiked-in template containing MAFs ranging from 50% down to 0.09% (Table 3, FIG. 3a). For 50% MAF to 1.6% MAF, there was excellent short term precision with a coefficient of variation <1% across all templates and a maximum Ct range (i.e. highest value Ct −lowest value Ct) of 0.72. At the more dilute templates with 0.78% MAF and 0.1% MAF there was slight loss of precision. The respective % CVs were 1.02 and 1.36 and Ct ranges were 1.10 and 1.38.

The templates had been diluted twofold and so the mean Ct value for each MAF was plotted against the log 2 [1/MAF] to enable the dynamic range to be assessed. Over this range, the slope of the curve was 1.07 (FIG. 3b) demonstrating a PCR efficiency of 105% with an intercept at Ct=25.43. Thus the PCR remained efficient over a large range of MAF and, with this level of efficiency, HOT_ARMS PCR could probably be used for mutant allele quantification.

HOT_ARMS PCR Works on DNA Derived from Formalin Fixed Paraffin-Embedded (FFPE) Tissue

The HOT_ARMS primers were designed to amplify short fragments with a view to using the assay on DNA obtained from formalin-fixed paraffin-embedded (FFPE) tissue. FFPE tissue-derived DNA is notorious for being fragmented and often of poor quality. A total of 10 cases, with known mutations in KRAS codon 12/13, were tested by HOT_ARMS PCR. For each case, there was successful amplification with the mutation-specific primer (FIG. 4a). The experiment was extended by testing the samples in a blinded fashion by setting a panel (comprising HOT_ARMS 1-HOT_ARMS 4) to interrogate this hot spot. All samples were correctly genotyped including a case which was wild type (FIG. 4b, Table 4).

Further testing of BRAF V600E on 35 positive cases validated by high resolution melting analysis and KRAS on a further 32 positives cases validated by pyrosequencing all gave 100% concordance.

Combining LNA/GC Tag Improves Specificity of HOT_ARMS PCR

Data had shown that, of the different strategies, the optimal primer modification was a 10 base GC tag at the 5′ end of both primers. Although this produced an exquisitely sensitive assay, the ΔCt value between test sample and placental DNA became progressively smaller at low MAF. This prompted us to combine the 10 base GC tag with LNA incorporation in order to reduce the non-specific bleed through amplification. By incorporating an LNA at the 3′ mutation-specific base of the HOT_ARMS1 primers, we were able to abrogate non-specific amplification and no signal was detected even after 50 cycles (FIG. 5a). This allowed the interpretation of the assay to be a dichotomous “yes/no” depending the presence/absence of amplification. The cost of this was however reduced efficiency of the primers (Table 5). The thermal cycling parameters had to change to a touch-up reaction which included 20 cycles with a Ta of 60° C. followed by 30 cycles of Ta of 71° C. Comparison of the metrics used for short-term precision (FIG. 5b) and dynamic range (FIG. 5c) showed that, at all MAF, the coefficient of variation of the combined primer pair was increased and that the efficiency of the PCR over the range of 50%-0.09% MAF was reduced. The slope of the curve was 2.25 demonstrating a PCR efficiency of 113%.

3′LNA primers with tags can be used with nested PCR to increase efficiency. Increased specificity remains and 0.1% MAF detection can be achieved.

Discussion

The Amplification Refractory Mutation System (ARMS) was originally described as a means of genotyping single nucleotide variants (SNVs) without the need for formal sequencing [10]. It has subsequently been used for mutation detection in cancer and, since PCR is mutation-specific, it can be very sensitive. However, with standard ARMS PCR, there is still low-level base-pairing at the 3′ end between the mutation-specific primer and the wild-type sequence despite being mismatched. This allows mis-priming of wild-type DNA by the mutant primer with consequent non-specific amplification. Discriminating non-specific from specific amplification when MAF is low can be problematic.

In High Optimized Ta ARMS (HOT_ARMS) PCR, specificity of the PCR is increased by modifying the primers to raise the annealing temperature (Ta). The increased kinetic energy of the primers hugely reduces non-specific 3′ base-pairing. This specificity is achieved with a Ta ≥65° C. although the higher the Ta the greater the specificity. The primers can be modified in a number of ways and, in our hands, the best results were obtained by adding a 10 base GC tag onto each primer. With just this modification and a “working” Ta of 71° C., we demonstrated that HOT_ARMS PCR is an incredibly simple, robust and exquisitely sensitive method of detecting low frequency mutant alleles.

Thirteen different mutations, located in different genes and exons and including both SNVs and indel mutations, were tested. We believe we are the first group to use ARMS PCR for detection of indels and the results were comparable with detection of SNVs. The primers required minimal optimization and in fact, after the first two sets of primers, optimization was found to be not necessary as the primers worked “off the shelf”. The short term precision (reproducibility) of the assay was tested whilst there was slight increase in coefficient of variation at very low MAF (<1.56%), in most cases it was <1% as would be expected for an accurate real-time test [25-27]. Similarly, the PCR efficiency was 105% over the MAF range of 50%-0.09% MAF indicating a wide dynamic range and potentially mutant allele quantification.

The limit of detection of the 13 primer pairs ranged from 0.004% MAF for the best performing primer to 0.125% for the worst performing primer. This variation is to be expected as, apart from hydrogen-bonding between base pairs, other sequence-dependent factors will contribute to the stability of the primer-DNA duplex. Thus some mutation-specific primers will be innately more specific than other. However the robustness of the methodology is reflected in the fact that all of the primer pairs, with little or no optimisation had a limit of detection of 0.06% MAF. This is underlined further by the fact that tests can be multiplexed and, when tested on DNA derived from FFPE tissue, the mutations could be easily detected and tumours could be correctly genotyped when tested blind using HOT_ARMS PCR. cfDNA is usually of much higher quality than FFPE tissue-derived DNA, therefore HOT_ARMS PCR would easily work with cfDNA.

The need to identify low frequency tumour-derived mutant alleles in a pool of wild-type DNA is well described in molecular diagnostics. A number of methods of varying complexity have been established for detection of very low MAF each with its own utility and advantages/disadvantages (Table 7). Apart from setting a different cycling program and purchasing GC tagged primers, HOT_ARMS PCR does not differ from a standard real-time PCR i.e. this is a single closed-tube reaction which does not require extra mismatches, special probes, special enzymes or a nested protocol. It is much more sensitive than ARMS PCR and there is no other probe-free single stage closed tube method that comes even close to the most common limit of detection of 0.06% MAF. This system has a similar limit of detection as other more complicated systems published in the literature. Addition of probes has been used to improve the limit of detection and some closed-tube systems claim to have a limit of detection as low as 0.005% MAF [3]. Probe-based methods will however be more complex than HOT_ARMS PCR and will not be universally applicable since each mutation will require manufacture of specific probes. Methods such as COLD-PCR and its derivatives [6] can enrich low frequency mutant alleles allowing detection down to a MAF of 0.1%. These do however require a second step (i.e. sequencing or mutation screening) in order to confirm the presence of mutations. There are other high throughput methods described such as Beaming, digital droplet PCR and ultra-deep Next Generation Sequencing, which have been reported to detect MAF as low as 0.001%. These however require expensive machine as well as complex manipulation of the template (such as generating libraries in NGS).

HOT_ARMS PCR requires prior knowledge of the sequence changes induced by mutations and thus it cannot be used for mutation screening unless there is a very limited spectrum of sequence change (such as with KRAS codon 12/13 mutation [36]). However, it is likely that, in the near future, all tumours will undergo either whole genome or whole exome sequencing and a full mutation profile will be described in each case. HOT_ARMS PCR can be used to detect any mutation which causes a sequence change including SNVs and indels. If the specific sequence changes can be identified in structural variants, it could be used to test for these too. As we have shown, HOT_ARMS PCR is readily applicable to all mutations and therefore patient specific primer sets can be established for tumour surveillance as soon as the mutation profile is known. Tumour surveillance may become a major part of the cancer care pathway especially in the wake of data showing that tumour specific mutations can be detected in the cfDNA of patients up to a year before recurrence becomes clinically overt. Since HOT_ARMS PCR can be undertaken within the two hours and does not require complex data interpretation, it could provide a result within the time scale of a hospital outpatient appointment. Equally feasible, would be the establishment of a patient-specific HOT_ARMS PCR tumour surveillance assays in the primary care setting.

In summary, HOT_ARMS PCR is an extremely simple, robust and exquisitely sensitive test for detection of any kind of mutation which results in a sequence change. It is a single stage closed-tube test which does not require expensive equipment and, because it is not reliant on probes, it is easy to set up and requires little optimisation for most mutations. The speed of the test means it could be established in the hospital out-patient and even the primary care setting.

### EXAMPLE 2—HIGHLY OPTIMISED ANNEALING TEMPERATURE—PROBEINHIBITED—POLYMERASE CHAIN REACTION (HOT_PI-PCR) TECHNICAL WHITE PAPER

Summary

We reasoned that issues with standard wild-type blocking PCR could be counteracted by increasing the primer annealing temperature (Ta). The increase in free energy generated by raising the annealing temperature to >65° C. would prevent inappropriate base-pairing and perfect base matches would be more stable resulting in a probe which had higher specificity. Hence, giving more freedom for the mutant DNA to amplify without probe mispriming and blocking the wild-type DNA further as the probe would bind with greater affinity. Moreover, raising the annealing temperature would also allow a larger probe, due to the 8° C. gap required, in turn generating a larger scanning region for mutations. Here we show that by a simple modification of the primers to increase the Ta ≥65° C., the specificity of wild-type blocking PCR can be increased and further fold enrichment can be generated compared to previous iterations of the system. Moreover, a scanning region of 50 bp can be achieved. We call this modification High Optimized Ta-Probe inhibited (HOT_PI) PCR.

Materials and Methods

DNA Extraction

DNA from was extracted from cell lines in accordance with Example 1.

DNA Dilutions and Limit of Detection

Placental DNA and some of the mutation-containing cell line DNA was purchased from commercial sources. All DNA was quantified using a Nanodrop spectrophotometer 2000c (Thermo Scientific) and 260:280 absorbance ratio of 1.8-2.0 was taken as indicative of good quality. DNA was diluted to a final concentration of 20 ng/μl with nuclease free water (Qiagen, Germany). DNA from cell lines with known mutations was spiked into placental DNA containing wild type sequence. Templates samples were prepared containing mutant alleles frequencies (MAF) ranging from 50% down to 0.1%.

Primer Design and Modification

(a) Primer Design

In total, 7 different mutations were tested in 2 different codons in 1 gene (see Table 1). The mutations tested in KRAS were single nucleotide variants.

HOT_PI PCR works on the principle that a high Ta will improve the specificity of the probe. As a rule of thumb, the Ta is usually 5° C. lower that the melting temperature (Tm). The definition of Tm is the temperature at which 50% of the DNA is melted (i.e. the strands are separated) and this is the physical property DNA used by primers design software packages. Primers were initially designed in Primer 3 [15] mostly according to the standard rules [16] and then modifications were made to raise the Tm/Ta.

A design guide for HOT_PI PCR is as follows: (i) minimum primer length 20 nt, optimum primer length 25 nt and maximum primer length 30 nt; (ii) minimum GC content 30%, optimum GC content 45%, maximum GC content 60%; (iii) minimum Tm (melting temperature) 60° C., optimum Tm 65° C., maximum Tm 85° C.; (iv) minimum amplicon length 50 nt, maximum amplicon length 110 nt; (v) primer dimer ΔG<−6 and Tm<60° C.; (vi) hairpin ΔG<−6 and Tm<60° C.; (vii) Max Tm difference between forward and reverse primer 3° C.

(b) Primer Modification

For HOT_PI PCR, the primers need to be modified to increase the Tm. There are two main approaches available i.e. addition of a sequence tag to the primers or incorporation of modified bases into the primers.

(i) Addition of a tag. Extending the length of a primer by adding extra sequences in the form of a 5′ tag will raise the Tm of the primer. The greatest increase will be achieved if the tag contains a high proportion of Guanine and Cytosine bases. We have previously added tags to primers in order to develop multiplexed HRM protocols [8, 17]. One tag was tested: a 10 base tag with the sequence 5′-gggccggccc-3′ (predicted to raise the Tm by approximately 20° C.). The tags were added to both the forward and reverse primers.

(ii) Incorporation of modified nucleic acids. Recently developed synthetic nucleic acids (such as Locked Nucleic Acids (LNA), Bridged Nucleic Acids (BNA) and Peptide Nucleic Acids (PNA)) have a much greater binding affinity for the paired base on the opposite DNA strand than naturally occurring nucleic acids [18-20]. Incorporation of synthetic nucleic acids into primers/probes has been used to increase the specificity [18, 21, 22] although the high binding affinity stabilizes double stranded DNA thereby increasing the Tm (by approximately 5° C. per nucleotide). Only LNA's were tested here (although BNA/PNA would be predicted to have the same effect). Primers were designed in accordance with the rules above and 3-5 LNAs were included at various sites within the primer in accordance with published recommendations [18, 21-23].

(c) Probe design. The probe was designed to cover the whole region of interest generated between the two primers and to also extend 5 bp into each primer binding site. 6 LNA bases were added to the probe in this example to improve binding affinity further and clamp the region. Further LNA bases, such as up to 10 have also been shown to improve binding and clamping. 3′ phosphate was added to prevent polymerase extension. The probe contained LNA bases on the hotspot regions of codon 12 and 13. LNAs were added solely to improve clamping of the region rather than to increase mismatch temperature.

HOT_PI PCR Protocol and Optimisation

All reactions were undertaken in 0.2 ml tubes with caps (Agilent, U.S.A). Each reaction was performed in a final volume of 10 μl which contained the following components: 2× HotShot Diamond mastermix (Clent Life Science, U.K) which includes a final concentration of 6 mM MgCl₂ and 400 μM dNTPs with stabiliser; EvaGreen dye 20× in water (Biotium, U.S.A); 100 nM LNA wild-type blocking probe with 3′ phosphate to prevent extension (Eurogentec); adjusted DNA template; 375 nM each primer and Nuclease-Free water (Qiagen, Germany). Standard nucleic acid primers were purchased from Eurofins (Luxembourg) and LNA probes were purchased from Eurogentec, (Belgium).

Primers were designed to have a minimum Tm of 70° C. to allow a Ta≥65° C. to be used. The initial optimisation used template containing 50% mutant alleles and a single PCR product was confirmed by high resolution melting analysis on a Lightscanner (Biofire Defence, U.S.A). In order to produce a standard protocol which would work for multiple targets, generally a Ta of 70° C. was used.

All tests were performed on the Stratagene MX3005P real-time machine. The threshold for detection was set using the machine's default parameters (10 standard deviations away from the mean of the baseline fluorescence). For tagged primers, template containing different mutant allele frequencies was tested using the following cycling parameters: (95° C./5 min)×1/(95° C./30 sec; Ta/15 sec; 72° C./15 sec)×10/(95° C./30 sec; 75° C./30 sec)×40/(72° C./5 min)×1. Similarly, fast cycling PCR for wild-type DNA amplification was carried out using the following protocol, demonstrating the potential speed of PCR using tagged primers (95° C./5 min)×1/(95° C./1 sec; 71/5 sec; 72° C./5 sec)×10/(95° C./1 sec; 75° C./5 sec)×30.

For sequencing, the PCR products were first purified using the GenElute PCR Clean-Up Kit (Sigma-Aldrich, Dorset, United Kingdom). The purified products were then diluted to 1-3 ng/ml following quantification in a NanoDrop 2000 UV spectrophotometer (Thermo Fisher Scientific). Sequencing was performed with the dye terminator chemistry (BigDye, version 3.1) on the 3130xl ABI PRISM Genetic Analyzer (Thermo Fisher Scientific). The sequencing data were viewed and analyzed using FinchTV software.

HRM and analysis were performed on the LightScanner96 Hi-Res Melting System (BioFireDiagnostics, Salt Lake City, Utah, USA). The PCR products were first transferred into a LightScanner 96-well hard-shell plate (Bio-Rad Laboratories, Hertfordshire, United Kingdom), followed by the addition of a 20 μl mineral oil overlay. Before HRM, plates were spun down in a Megafuge centrifuge (2500 rpm, 5 min; ThermoFisherScientific, Winsford, United Kingdom). HRM was performed between 65 and 95° C. with sample equilibration at 62° C. Exposure was set to“auto,” and data were captured at a ramp rate of 0.1° C./s. The acquired melting data were analyzed with the LightScanner Call-IT software, version 2.0.0.1.331.

HOT_PI PCR Assay Testing and Statistical Analysis

Limit-of-detection tests were performed on templates containing varying proportions of mutant allele and compared against HEK293T DNA (containing 0% mutant allele). In general, 40 ng of template was used with the lowest number of mutant copies being 8 (0.1%). When testing the performance of HOT_PI PCR on DNA derived from FFPE tissue, 40 ng of tumour DNA was used.

### EXAMPLE 3—HIGHLY OPTIMISED ANNEALING TEMPERATURE—POLYMERASE CHAIN REACTION

Summary

We reasoned that issues with PCR could be counteracted by increasing the primer annealing temperature (Ta). The increase in free energy generated by raising the annealing temperature to ≥65° C. would prevent inappropriate base-pairing and perfect base matches would be more stable resulting in increased oligonucleotide specificity as proven with invention example 1 and 2. Moreover, we reasoned that the implementation of 5′ tags can increase selectivity for PCR amplicons over DNA template, reducing the amount of non-specific amplification in PCR, especially in exponential phase. When tagged primers initially bind DNA, the tag can only partially bind DNA as it does not contain the tag sequence. When primers form amplicons they are incorporated. Thus, when amplicons are formed, the tag sequence is present in the template for primers to bind with perfect complementarity rather than partial complementarity and this causes further gains in the maximum annealing temperature. This novel finding generates the ability to generate a 2-phase touch-up cycling PCR with phase 1 having a lower maximum potential annealing temperature than phase 2. During phase 2 the raised annealing temperature which is beyond the annealing temperature that can be achieved in phase 1 will provide preferential amplification of amplicons rather than DNA. For example, in cycle 1 as amplicons are forming, the maximum annealing temperature may be 71° C. After cycle 1, the annealing temperature may be increased to 72-80° C. Since polymerase activity is between 68-80° C., extension can still occur. Cycling can alternate between 95° C. and 72-80° C. and this greatly reduces the amount of time spent ramping up and down to standard annealing temperatures of 45-60° C. Moreover, PCR can be carried out using standard 1-phase PCR at >65° C. and still result in increased selectivity whereby tagged primers after cycle 2 bind amplicons preferentially due to tag incorporation, creating perfect complementarity. Dramatic increases in specificity result in less failure of PCR; reduced formation of non-specific products; increased multiplexing capability and increased amplification of areas of the genome containing difficult template which has high similarity with other sequences. We call this modification High Optimized Ta-PCR.

Materials and Methods

DNA Extraction

DNA from was extracted from cell lines in accordance with Example 1.

DNA Dilutions and Limit of Detection

All DNA was quantified using a Nanodrop spectrophotometer 2000c (Thermo Scientific) and 260:280 absorbance ratio of 1.8-2.0 was taken as indicative of good quality.

HEK293T cell line DNA was diluted with nuclease free water (Qiagen, Germany) to 120 ng/μl, 80 ng/μl, 60 ng/μl, 40 ng/μl, 20 ng/μl, 10 ng/μl, 5 ng/μl and 1 ng/μl, 100 μg/μl.

Primer Design and Modification

(a) Primer Design

In total, 2 exons in 2 genes were tested; KRAS (HOT_PI primers) and EGFR (HOT_WT13/HOT_ARMS13 (wild-type) (see Table 1 for primers).

HOT_PCR works on the principle that a high Ta will improve the specificity and speed of the PCR. As a rule of thumb, the Ta is usually 5° C. lower that the melting temperature (Tm). The definition of Tm is the temperature at which 50% of the DNA is melted (i.e. the strands are separated) and this is the physical property DNA used by primers design software packages. Primers were initially designed in Primer 3 [15] mostly according to the standard rules [16] and then modifications were made to raise the Tm/Ta.

A design guide for HOT_PCR is as follows: (i) minimum primer length 20 nt, optimum primer length 25 nt and maximum primer length 30 nt; (ii) minimum GC content 30%, optimum GC content 45%, maximum GC content 60%; (iii) minimum Tm (melting temperature) 60° C., optimum Tm 65° C., maximum Tm 85° C.; (iv) minimum amplicon length 50 nt, maximum amplicon length 110 nt; (v) primer dimer ΔG<−6 and Tm<60° C.; (vi) hairpin ΔG<−6 and Tm<60° C.; (vii) Max Tm difference between forward and reverse primer 3° C.

(b) Primer Modification

For HOT_PCR, the primers need to be modified to increase the Tm. There are two main approaches available i.e. addition of a sequence tag to the primers or incorporation of modified bases into the primers.

(i) Addition of a tag. Extending the length of a primer by adding extra sequences in the form of a 5′ tag will raise the Tm of the primer. The greatest increase will be achieved if the tag contains a high proportion of Guanine and Cytosine bases. We have previously added tags to primers in order to develop multiplexed HRM protocols [8, 17]. One tag was tested: a 10 base tag with the sequence 5′-gggccggccc-3′ (predicted to raise the Tm by approximately 20° C.). The tags were added to both the forward and reverse primers.

(ii) Incorporation of modified nucleic acids. Recently developed synthetic nucleic acids (such as Locked Nucleic Acids (LNA), Bridged Nucleic Acids (BNA) and Peptide Nucleic Acids (PNA)) have a much greater binding affinity for the paired base on the opposite DNA strand than naturally occurring nucleic acids [18-20]. Incorporation of synthetic nucleic acids into primers/probes has been used to increase the specificity [18, 21, 22] although the high binding affinity stabilizes double stranded DNA thereby increasing the Tm (by approximately 5° C. per nucleotide). Only LNA's were tested here (although BNA/PNA would be predicted to have the same effect). Primers were designed in accordance with the rules above and 3-5 LNAs were included at various sites within the primer in accordance with published recommendations [18, 21-23].

HOT_PCR Protocol and Optimisation

All reactions were undertaken in 0.2 ml tubes with caps (Agilent, U.S.A). Each reaction was performed using fast cycling mastermixes in a final volume of 10 μl which contained the following components: 2× fast cycling PCR mastermix (Qiagen, Germany) or 2× sensiFAST HRM (Bioline, England); EvaGreen dye 20× in water (Biotium, U.S.A); adjusted DNA template; 375 nM each primer and Nuclease-Free water (Qiagen, Germany). Standard nucleic acid primers were purchased from Eurofins (Luxembourg) and LNA primers were purchased from Eurogentec, (Belgium).

Primers were designed to have a minimum Tm of 70° C. to allow a Ta≥65° C. to be used. The initial optimisation used template containing 40 ng total wild-type DNA and a single PCR product was confirmed by high resolution melting analysis on a Lightscanner (Biofire Defence, U.S.A). In order to produce a standard protocol which would work for all targets universally, generally a Ta of 66° C. was used for maximum efficiency. All tests were performed on the Stratagene MX3005P real-time machine. The threshold for detection was set using the machine's default parameters (10 standard deviations away from the mean of the baseline fluorescence). For tagged primers, template containing different amounts of total DNA was tested using the following cycling parameters: (95° C./5 min)×1/(95° C./5 sec; 66° C./5 sec; 68° C./5 sec)×10/(95° C./5 sec; 70° C./10 sec)×30/(72° C./1 min)×1. Using standard PCR machines with regular ramp rates, PCR can be completed in 26 minutes. This would be enhanced further with fast ramping PCR machines.

HOT_PCR Assay Testing

Specificity increases for singleplex and multiplex PCR have been proven in example 1 and 2 as well as checking for absence of effects the modification may have on amplifying low amounts of cell line DNA, FFPE DNA and cell-free DNA. Thus, amplification of a range of total HEK293T DNA was tested to determine reliability and detection of low level DNA input when undergoing rapid amplification.

Tables

TABLE 1
Sequence of primers for HOT_ARMS and wild-type positive control primers,
HOT_WT. Only the 3′ base differs between HOT_ARMS and HOT_WT. Moreover, they
both pair with the same common wild-type primer. For example: HOT_ARMS 1
(mutation specific primer) pairs with HOT_ARMS 1 (wild-type primer) to
detect mutation specific sequence. HOT_WT1 (wild-type primer) pairs with
HOT_ARMS 1 (wild-type primer) to detect wild-type specific sequence as
a positive control. This method allows individual quantification of the
mutant sequence and wild-type sequence separately rather than quantification
of total DNA (mutant and wild-type combined). Therefore, in a scenario
where a sample is 100% mutant, the wild-type positive control primer pair
will not generate a signal and the ARMS primer pair will generate a
strong signal giving further validation for the result.

| | | ARMS/HOT_WT | | | | Amplicon |
|---|---|---|---|---|---|---|
| | | or wild type | Target | Sequence | | length |
| Ref | Direction | primer | gene | change | Primer sequence (5′ to 3′) | (bp) |
| HOT_ARMS1 | Forward | ARMS | KRAS | c.38G > A | CTTGTGGTAGTTGGAGCTGGTGA | 60 |
| | | | | | (SEQ ID NO: 1) | |
| HOT_ARMS2 | Reverse | ARMS | KRAS | c.34C > A | GCACTCTTGCCTACGCCACA | 60 |
| | | | | | (SEQ ID NO: 2) | |
| HOT_ARMS2b | Forward | ARMS | KRAS | c.34G > T | TATAAACTTGTGGTAGTTGGAGCTT | 66 |
| | | | | | (SEQ ID NO: 3) | |
| HOT_ARMS3a | Reverse | ARMS | KRAS | c.35C > A | GCACTCTTGCCTACGCCAA | 60 |
| | | | | | (SEQ ID NO: 4) | |
| HOT_ARMS3b | Forward | ARMS | KRAS | c.35G > T | ATAAACTTGTGGTAGTTGGAGCTGT | 65 |
| | | | | | (SEQ ID NO: 5) | |
| HOT_ARMSS4 | Forward | ARMS | KRAS | c.35G > A | ATAAACTTGTGGTAGTTGGAGCTGA | 65 |
| | | | | | (SEQ ID NO: 6) | |
| HOT_ARMSS5 | Forward | ARMS | KRAS | c.34G > C | TATAAACTTGTGGTAGTTGGAGCTC | 66 |
| | | | | | (SEQ ID NO: 7) | |
| HOT_ARMS6 | Forward | ARMS | KRAS | c.34G > A | TATAAACTTGTGGTAGTTGGAGCTA | 66 |
| | | | | | (SEQ ID NO: 8) | |
| HOT_ARMS7 | Reverse | ARMS | KRAS | c.35C > G | GCACTCTTGCCTACGCCAG | 60 |
| | | | | | (SEQ ID NO: 9) | |
| HOT_ARMS8 | Forward | ARMS | PIK3CA | c.1624G > A | GCAATTTCTACACGAGATCCTCTCTCTA | 65 |
| | | | | | (SEQ ID NO: 10) | |
| HOT_ARMS9 | Forward | ARMS | PIK3CA | c.1633G > A | GAGATCCTCTCTGAAATCACTA | 69 |
| | | | | | (SEQ ID NO: 11) | |
| HOT_ARMS10 | Forward | ARMS | PIK3CA | c.3140A > G | CATGAAACAAATGAATGATGCACG | 80 |
| | | | | | (SEQ ID NO: 12) | |
| HOT_ARMS11 | Forward | ARMS | APC | c.4287_4296 | TGATCTTCCAGATAGCCCTGGACAC | 83 |
| | | | | delAACCATGC | (SEQ ID NO: 13) | |
| | | | | CA | | |
| HOT_ARMS12 | Reverse | ARMS | BRAF | c.1799A > T | GGACCCACTCCATCGAGATTTCT | 70 |
| | | | | | (SEQ ID NO: 14) | |
| HOT_ARMS13 | Reverse | ARMS | EGFR | c.2369C > T | CGAAGGGCATGAGCTGCA | 50 |
| | | | | | (SEQ ID NO: 15) | |
| HOT_WT2b, | Forward | HOT_WT | KRAS | c.34 | TATAAACTTGTGGTAGTTGGAGCTG | 66 |
| 5, 6 | | | | | (SEQ ID NO: 16) | |
| HOT_WT2a | Reverse | HOT_WT | KRAS | c.34 | GCACTCTTGCCTACGCCACC | 60 |
| | | | | | (SEQ ID NO: 17) | |
| HOT_WT3b, | Forward | HOT_WT | KRAS | c.35 | ATAAACTTGTGGTAGTTGGAGCTGG | 65 |
| 4 | | | | | (SEQ ID NO: 18) | |
| HOT_WT3a, | Reverse | HOT_WT | KRAS | c.35 | GCACTCTTGCCTACGCCAC | 60 |
| 7 | | | | | (SEQ ID NO: 19) | |
| HOT_WT1 | Forward | HOT_WT | KRAS | c.38 | CTTGTGGTAGTTGGAGCTGGTGG | 60 |
| | | | | | (SEQ ID NO: 20) | |
| HOT_WT8 | Forward | HOT_WT | PIK3CA | c.1624 | GCAATTTCTACACGAGATCCTCTCTG | 65 |
| | | | | | (SEQ ID NO: 21) | |
| HOT_WT9 | Forward | HOT_WT | PIK3CA | c.1633 | GAGATCCTCTCTGAAATCACTG | 69 |
| | | | | | (SEQ ID NO: 22) | |
| HOT_WT10 | Forward | HOT_WT | PIK3CA | c.3140 | CATGAAACAAATGAATGATGCACA | 80 |
| | | | | | (SEQ ID NO: 23) | |
| HOT_WT11 | Forward | HOT_WT | APC | c.4287 | TGATCTTCCAGATAGCCCTGGACAA | 93 |
| | | | | | (SEQ ID NO: 24) | |
| HOT_WT12 | Forward | HOT_WT | BRAF | c.1799 | GGACCCACTCCATCGAGATTTCA | 70 |
| | | | | | (SEQ ID NO: 25) | |
| HOT_WT13 | Reverse | HOT_WT | EGFR | c.2369 | CGAAGGGCATGAGCTGCG | 50 |
| | | | | | (SEQ ID NO: 26) | |
| HOT_ARMS1, | Reverse | Wild type | KRAS | N/A | CTGAATTAGCTGTATCGTCAAGGCA | N/A |
| 2b, 3b, | | | | | (SEQ ID NO: 27) | |
| 4, 5, 6 | | | | | | |
| HOT_ARMS2a, | Forward | Wild type | KRAS | N/A | GCTGAAAATGACTGAATATAAACTTGTGGT | |
| 3a, 7 | | | | | (SEQ ID NO: 28) | N/A |
| HOT_ARMS8 | Reverse | Wild type | PIK3CA | N/A | TGACTCCATAGAAAATCTTTCTCCTGCT | N/A |
| | | | | | (SEQ ID NO: 29) | |
| HOT_ARMS9 | Reverse | Wild type | PIK3CA | N/A | ATTTTAGCACTTACCTGTGACTC | N/A |
| | | | | | (SEQ ID NO: 30) | |
| HOT_ARMS10 | Reverse | Wild type | PIK3CA | N/A | CATGCTGTTTAATTGTGTGGAAGA | N/A |
| | | | | | (SEQ ID NO: 31) | |
| HOT_ARMS11 | Reverse | Wild type | APC | N/A | ACTTCTCGCTTGGTTTGAGCTGTTT | N/A |
| | | | | | (SEQ ID NO: 32) | |
| HOT_ARMS12 | Forward | Wild type | BRAF | N/A | TCATGAAGACCTCACAGTAAAAATAGGT | N/A |
| | | | | | (SEQ ID NO: 33) | |
| HOT_ARMS13 | Forward | Wild-type | EGFR | N/A | CATCTGCCTCACCTCCACCG | N/A |
| | | | | | (SEQ ID NO: 34) | |

Table 2 Performance of HOT_ARMS PCR in multiple assays. Comparison of ΔCt values for a commercial mastermix. Demonstrates HOT_ARMS performance using a mastermix without special conditions or enhancers.

TABLE 2
Thirteen different primer pairs were designed for a number of different targets and this table shows
the comparison between the relevant mutant cell lines and wild-type DNA. The column labelled LOD shows
the limit of detection. HOT_ARMS1 was tested to its absolute limit. The remainder were not tested
to limit to limit as this was felt to be sufficient. (MAF = mutant allele frequency).

| | | | | Commercial | |
|---|---|---|---|---|---|
| | | | | mastermix | |
| | | | | 40 ng total | |
| | | Protein | | DNA − ΔCt | |
| | | sequence/ | | 50% MAF vs | Sensitivity |
| Ref | Direction | common name | Sequence change | wild-type | achieved |
| HOT_ARMS1 | Forward | KRAS G13D | c.38G > A | 12.395 | 0.004% |
| HOT_ARMS2a | Reverse | KRAS G12C | c.34C > A | >24.955 | 0.01% |
| HOT_ARMS3a | Reverse | KRAS G12V | c.35C > A | >24.385 | 0.01% |
| HOT_ARMS4 | Forward | KRAS G12D | c.35G > A | 10.665 | 0.06% |
| HOT_ARMS5 | Reverse | KRAS G12R | c.34C > G | >24.655 | 0.01% |
| HOT_ARMS6 | Forward | KRAS G12S | c.34G > A | 13.395 | 0.06% |
| HOT_ARMS7 | Reverse | KRAS G12A | c.35C > G | 16.18 | 0.01% |
| HOT_ARMS8 | Forward | PIK3CA | c.1624G > A | 16.15 | 0.01% |
| | | E542K | | | |
| HOT_ARMS9 | Forward | PIK3CA | c.1633G > A | 11.825 | 0.06% |
| | | E545K | | | |
| HOT_ARMS10 | Forward | PIK3CA | c.3140A > G | 12.315 | 0.06% |
| | | H1047R | | | |
| HOT_ARMS11 | Forward | APC | c.4287_4296delAACCATGCCA | 14.67 | 0.06% |
| | | p.Q1429fs*41 | | | |
| HOT_ARMS12 | Reverse | BRAF | c.1799A > T | >22.87 | 0.01% |
| | | V600E | | | |
| HOT_ARMS13 | Reverse | EGFR | c.2369C > T | 12.37 | 0.06% |
| | | T790M | | | |

TABLE 3
HOT_ARMS PCR short term precision.
Table 3. HOT_ARMS1 primers were tested for their short term precision.
Cell line DNA was spiked into wild-type DNA to produce mutant allele
frequency (MAF) ranging from 50%-0.09%. Eight replicates were
performed and the results of the test are shown. The coefficient of
variation (CV %) was below 1% until the MAF reached 0.78% indicating
that the test is robust of a wide range.

| MAF (%) | 50.00 | 25.00 | 12.50 | 6.25 | 3.13 | 1.56 | 0.78 | 0.09 |
|---|---|---|---|---|---|---|---|---|
| Mean | 26.26 | 27.45 | 28.72 | 29.84 | 31.03 | 31.85 | 33.37 | 35.79 |
| Min | 26.18 | 27.04 | 28.47 | 29.5 | 30.77 | 31.52 | 32.79 | 35.02 |
| Max | 26.42 | 27.68 | 28.99 | 30.16 | 31.24 | 32.24 | 33.89 | 36.4 |
| Range | 0.24 | 0.64 | 0.52 | 0.66 | 0.47 | 0.72 | 1.1 | 1.38 |
| CV % | 0.30 | 0.74 | 0.58 | 0.76 | 0.51 | 0.92 | 1.02 | 1.36 |

TABLE 4
Testing of HOT_ARMS PCR for KRAS exon 2 codons 12 and 13 mutations on DNA from formalin-fixed tissue.
Table 4. The HOT_ARMS PCR was tested on 10 previously genotyped samples derived from formalin-fixed tissue.
The samples were initially tested for their known mutations and these were confirmed. They were then
tested “blind” using HOT_ARMS1-4 as a panel and each sample was correctly genotyped. Sample 4 was
classed as unknown due to mutation being detected by HRM but not confirmed by Sanger Sequencing.
The HOT_ARMS data shows that this is probably a c38G > A mutation but the relatively high Ct value
indicates it is present at a low frequency.

| Sample | 1 | 2 | 3 | 4 | 5 | 6 | 7 | 8 | 9 | 10 |
|---|---|---|---|---|---|---|---|---|---|---|
| Validated | C35G > T | C34G > T | C34G > T | Unknown | C38G > A | C35G > A | None | C35G > A | C35G > T | C35G > A |
| mutation | | | | | | | | | | |
| (Sanger | | | | | | | | | | |
| sequencing) | | | | | | | | | | |
| HOT_ARMS1 | 35.69 | 37.23 | 38.96 | 32.87 | 27.26 | 37.44 | 37.79 | 35.33 | 37.64 | 36.76 |
| C38G > A | | | | | | | | | | |
| ct value | | | | | | | | | | |
| HOT_ARMS2b | 36.69 | 29.35 | 27.64 | 35.7 | 34.53 | 35.63 | 36.41 | 35.06 | 35.58 | 34.45 |
| C34G > T | | | | | | | | | | |
| ct value | | | | | | | | | | |
| HOT_ARMS3b | 28.33 | 34.84 | 34.89 | 37.24 | 36.74 | 35.22 | 35.96 | 35.43 | 30.88 | 38.15 |
| C35G > T | | | | | | | | | | |
| ct value | | | | | | | | | | |
| HOT_ARMS4 | 34.79 | 35.98 | 35.43 | 34.75 | 34.45 | 28.97 | 37.15 | 25.9 | 35.12 | 29.59 |
| C35G > A | | | | | | | | | | |
| ct value | | | | | | | | | | |
| Genotyping | C35G > T | C34G > T | C34G > T | C38G > A | C38G > A | C35G > A | None | C35G > A | C35G > T | C35G > A |
| result | | | | | | | | | | |
| (HOT_ARMS) | | | | | | | | | | |

TABLE 5
Short term precision of HOT_ARMS PCR
with combined GC-rich tag and 3′LNA
Table 5. HOT_ARMS1 primers were modified to include a
locked nucleic acid (LNA) at the 3′ base of the
mutation specific primer. This abrogated amplification from
placental DNA (even after 50 cycles) but there was reduced
efficiency. Cell line DNA was spiked into placental DNA
to produce mutant allele frequency (MAF) ranging from
50%-0.09%. Eight replicates were performed and the results
of the test are shown. The coefficient of variation (CV %)
was increased compared to the GC tag alone.

| MAF(%) | 50.00 | 25.00 | 12.50 | 6.25 | 3.13 | 1.56 | 0.78 | 0.10 |
|---|---|---|---|---|---|---|---|---|
| Mean | 27.53 | 30.34 | 33.65 | 36.27 | 38.42 | 40.66 | 42.89 | 47.59 |
| Min | 27.32 | 29.73 | 32.66 | 35.28 | 37.50 | 39.19 | 40.50 | 45.05 |
| Max | 28.18 | 30.81 | 34.45 | 37.25 | 39.14 | 41.43 | 44.82 | 49.76 |
| Range | 20.86 | 21.08 | 21.79 | 21.97 | 21.64 | 22.24 | 24.32 | 24.71 |
| CV % | 3.85 | 3.60 | 4.11 | 4.10 | 3.41 | 3.55 | 6.72 | 5.81 |

TABLE 6
Demonstrates easy detection of low copy number with lower amounts of
total DNA input. Circulating tumour DNA produces low yield; hence, performance
may be enhanced by performing PCR in replicates with lower total DNA
or by screening for multiple targets with lower total DNA.

| | | | | 5 ng total |
|---|---|---|---|---|
| | | | | DNA − ΔCt |
| | | Protein | | 4 mutant |
| | | sequence/ | | copies vs |
| Ref | Direction | common name | Sequence change | wild-type |
| HOT_ARMS1 | Forward | KRAS G13D | c.38G > A | 5.56 |
| HOT_ARMS2a | Reverse | KRAS G12C | c.34C > A | 10.065 |
| HOT_ARMS3a | Reverse | KRAS G12V | c.35C > A | >13.12 |
| HOT_ARMS4 | Forward | KRAS G12D | c.35G > A | 5.51 |
| HOT_ARMS5 | Reverse | KRAS G12R | c.34C > G | 8.70 |
| HOT_ARMS6 | Forward | KRAS G12S | c.34G > A | 2.35 |
| HOT_ARMS7 | Reverse | KRAS G12A | c.35C > G | 9.46 |
| HOT_ARMS8 | Forward | PIK3CA E542K | c.1624G > A | 9.28 |
| HOT_ARMS9 | Forward | PIK3CA E545K | c.1633G > A | 1.97 |
| HOT_ARMS10 | Forward | PIK3CA H1047R | c.3140A > G | 3.97 |
| HOT_ARMS11 | Forward | APC | c.4287_4296delAACCATGCCA | 4.85 |
| | | p.Q1429fs*41 | | |
| HOT_ARMS12 | Reverse | BRAF V600E | c.1799A > T | 11.41 |
| HOT_ARMS13 | Reverse | EGFR T790M | c.2369C > T | 2.11 |

TABLE 7
Demonstration of wild-type blocking probe addition to
HOT_ARMS PCR to further enhance ΔCt values.

| | | ΔCt 50% | ΔCt 0.1% | Ct: | Ct: | |
|---|---|---|---|---|---|---|
| | Blocker | MAF & | MAF & | 50% | 0.1% | Ct: |
| Target | concentration | wild-type | wild-type | MAF | MAF | wild-type |
| HOT_ARMS4 | 0 nM | 10.665 | 1.405 | 26.435 | 35.695 | 37.100 |
| | 30 nM | 14.436 | 5.311 | 31.10 | 40.225 | 45.536 |
| HOT_ARMS6 | 0 nM | 13.395 | 2.855 | 25.980 | 36.520 | 39.375 |
| | 20 nM | 19.62 | 8.45 | 30.38 | 41.55 | 48.874 |

TABLE 8
Demonstration of HOT_ARMS12 (BRAF V600E) rapid testing. Highly
specific HOT_ARMS primers can take advantage of fast cycling
mastermixes and PCR machines (Magnetic induction cycling) to reduce
total cycling times to 30 minutes and maintain similar performance
as regular HOT_ARMS PCR. Also shown in FIG. 7.

| | MAF (40 ng total DNA) | | | | | | |
|---|---|---|---|---|---|---|---|
| | | | | | | | Wild- |
| | 50% | 25% | 12.5% | 6.25% | 1% | 0.06% | type |
| Ct value | 26.34 | 27.51 | 28.41 | 29.36 | 32.40 | 35.23 | 43.06 |

TABLE 9
Demonstration of HOT_ARMS multiplexing for multiple genes in the same
reaction. Table 5 represents multiplexing of HOT_ARMS1 (KRAS c.38G >
A) and HOT_ARMS12 (BRAF V600E) as well as individual singleplex reactions.
The individual mutation can be identified as well as yes/no calling
for mutations by melting peak analysis demonstrated in FIG. 8.

| | 50% | 1% | 0.1% | 50% | 1% | 0.1% | |
|---|---|---|---|---|---|---|---|
| | MAF | MAF | MAF | MAF | MAF | MAF | |
| | BRAF | BRAF | BRAF | KRAS | KRAS | KRAS | Wild- |
| Test | V600E | V600E | V600E | c.38G > A | c.38G > A | c.38G > A | type |
| Singleplex | | | | 26.07 | 38.1 | 39.49 | 42.42 |
| HOT_ARMS1 ct | | | | | | | |
| value | | | | | | | |
| Singleplex | 27.19 | 37.27 | 40.295 | | | | 48.14 |
| HOT_ARMS12 ct | | | | | | | |
| value | | | | | | | |
| Multiplex | 26.82 | 33.05 | 33.54 | 25.69 | 31.865 | 33.22 | 34.47 |
| HOT_ARMS1/12 | | | | | | | |

TABLE 10
Amplification of cfDNA, FFPE and cell line DNA with wild-type
modified primers. Demonstrates that the modification has no
effect on amplifying low DNA input fragmented DNA samples.

| | 2 ng | 1 ng | 250 pg | 250 pg | 125 pg | 125 pg | 62.5 pg | 62.5 pg |
|---|---|---|---|---|---|---|---|---|
| Sample | total | total | total | total | total | total | total | total |
| type | DNA ct | DNA ct | DNA ct | DNA ct | DNA ct | DNA ct | DNA ct | DNA ct |
| FFPE | 31.06 | 30.66 | 31.62 | 31.62 | 32.47 | 32.35 | 32.00 | 32.50 |
| DNA | | | | | | | | |
| sample | | | | | | | | |
| 1 | | | | | | | | |
| FFPE | 32.10 | 31.73 | 32.88 | 34.09 | 32.09 | 32.48 | 32.86 | 33.83 |
| DNA | | | | | | | | |
| sample | | | | | | | | |
| 2 | | | | | | | | |
| FFPE | 31.51 | 31.30 | 32.34 | 32.29 | 32.69 | 33.22 | 33.44 | 33.37 |
| DNA | | | | | | | | |
| sample | | | | | | | | |
| 3 | | | | | | | | |
| cfDNA | 31.94 | 32.40 | 32.39 | 33.02 | 33.12 | 33.66 | 33.36 | 32.53 |
| sample | | | | | | | | |
| 1 | | | | | | | | |
| cfDNA | 32.26 | 31.67 | 31.59 | 31.10 | 31.56 | 31.76 | 31.77 | 31.78 |
| sample | | | | | | | | |
| 2 | | | | | | | | |
| cfDNA | 30.33 | 30.58 | 30.96 | 30.77 | 31.55 | 30.42 | 32.81 | 32.10 |
| sample | | | | | | | | |
| 3 | | | | | | | | |
| Cell line | 29.67 | 30.22 | 30.44 | 30.62 | 31.79 | 30.54 | 31.70 | 34.22 |
| DNA | | | | | | | | |

TABLE 11
Comparison of HOT_ARMS2a screening results on cfDNA samples
with Qiagen's current Therascreen assay for KRAS G12C. Samples
were blinded by Qiagen before screening by HOT_ARMS.

| | | | HOT | | | |
|---|---|---|---|---|---|---|
| | | | Wild- | Qiagen | Mutation | Mutation |
| Sample | HOT_ARMS2a | Qiagen | type ct | WT Ct | status | status |
| name | ct value | mut Ct | value | value | HOT_ARMS2a | Qiagen |
| 1 | 38.46 | 34.31 | 29.02 | 27.53 | Mutant | Mutant |
| 2 | >50 | n/a | 50 | n/a | Wild | Wild |
| 3 | >50 | n/a | 31.23 | 30.14 | Wild | Wild |
| 4 | >50 | n/a | 30.62 | 28.55 | Wild | Wild |
| 5 | >50 | n/a | 29.78 | 28.85 | Wild | Wild |
| 6 | >50 | n/a | 29.91 | 28.26 | Wild | Wild |
| 7 | >50 | n/a | 30.31 | 29.19 | Wild | Wild |
| 8 | 38.87 | 35.89 | 30.09 | 29.34 | Mutant | Mutant |
| 9 | >50 | n/a | 26.89 | 25.61 | Wild | Wild |
| 10 | >50 | n/a | 31.04 | 29.98 | Wild | Wild |
| 11 | 37.01 | 36.31 | 29.49 | 28.43 | Mutant | Mutant |

TABLE 12
HOT_PI ct values for different wild-type blocking probe concentrations. 1% MAF
becomes easily visible by ct value using higher concentrations of wild-type blocking
probe. However, sequencing and high-resolution melting analysis require wild-type
DNA present for analysis and ct values of below 30. Mutant specific probes could
be combined with the technique for mutation detection with high sensitivity.

| | | | | | | Δct 1% |
|---|---|---|---|---|---|---|
| Wild-type blocking | c.35G > T | c.34G > A | c.38G > A | c.38 G > A | | and |
| probe | 100% | 100% | 50% | 1% | Wild- | wild- |
| concentration | MAF | MAF | MAF | MAF | type | type |
| 0 nM | 15.92 | 18.52 | 16.97 | 18.02 | 18.21 | 0.19 |
| 100 nM | 17.24 | 20.18 | 20.10 | 25.06 | 26.48 | 1.42 |
| 200 nM | 18.33 | 21.53 | 21.59 | 27.84 | 33.61 | 6.13 |
| 300 nM | 19.28 | 22.73 | 23.15 | 30.32 | 39.32 | 9.00 |

TABLE 13
Demonstration of HOT_PI PCR combined with HOT_ARMS PCR. HOT_PI PCR has
been used in the first stage reaction of a nested procedure and detection
has been carried out in a second stage reaction by HOT_ARMS1 (KRAS c.38 G > A).
Supreme sensitivity can be achieved with very low cross-reactivity with other
mutations (c.35 G > T and c.34G > A). Also shows potential for nested
HOT_ARMS PCR without HOT_PI enrichment as 1% MAF can still be easily detected.

| Wild-type blocking | c.35G > T | c.34G > A | c.38G > A | c.38 G > A | Wild- |
|---|---|---|---|---|---|
| probe concentration | 100% MAF | 100% MAF | 50% MAF | 1% MAF | type |
| 0 nM | 25.28 | 22.63 | 6.40 | 12.24 | 16.20 |
| 100 nM | 24.68 | 22.37 | 6.22 | 6.72 | 12.39 |

TABLE 14
Cell lines used for testing HOT_ARMS PCR. This table lists the cell lines used and the
mutations contained in them. Most of the cell lines were available in-house (obtained
from the NCI 60 panel) whilst some had to be obtained from external commercial sources.

| | | | Cell | | | Cell line |
|---|---|---|---|---|---|---|
| | Protein | | line | Cell line | | DNA |
| | sequence/ | | harbouring | tumour | | sourced |
| Gene | common name | Gene sequence | mutation | source | Zygosity | from |
| KRAS | p.G13D | c.38G > A | HCT116 | Colon | Heterozygous | In house |
| KRAS | p.G12C | c.34G > T | SW837 | Colon | Heterozygous | In house |
| KRAS | p.G12V | c.35G > T | SW480 | Colon | Homozygous | In house |
| KRAS | p.G12D | c.35G > A | GP2D | Colon | Heterozygous | In house |
| KRAS | p.G12R | c.34G > C | SW48 + | Colon + | Heterozygous | Commercial |
| | | | G12R | knockin | | |
| KRAS | p.G12A | c.35G > C | SW1116 | Colon | Heterozygous | Commercial |
| KRAS | p.G12S | c.34G > A | A549 | Lung | Homozygous | In house |
| PIK3CA | p.E542K | c.1624G > A | SW948 | Colon | Heterozygous | Commercial |
| PIKCA | p.E545K | c.1633G > A | MCF7 | Breast | Heterozygous | In house |
| PIK3CA | p.H1047R | c.3140A > G | HCT116 | Colon | Heterozygous | In house |
| BRAF | p.V600E | c.1799 T > A | HT-29 | Colon | Heterozygous | In house |
| TP53 | p.R273H | c.818G > A | HT-29 | Colon | Homozygous | In house |
| APC | p.T1556fs*3 | c.4666_4667insA | HT-29 | Colon | Heterozygous | In house |
| APC | p.Q1429fs*41 | c.4287_4296delAACCATGCCA | SW1116 | Colon | Heterozygous | Commercial |

TABLE 15
Methods for detecting low frequency mutations. Table 7. This is a comparative
analysis of the different methods available for testing for low mutant allele frequency
and how they compare with HOT_ARMS PCR. Closed-tube means a single test
whilst open tube means that at least two tests are required.

| | | | Closed or | Time to | | |
|---|---|---|---|---|---|---|
| Technique | Sensitivity | Technical ease and cost | open tube | process | | Reference |
| Hot_ARMS | 0.004- | Real time PCR machine | Closed | 1 | day | N/A |
| | 0.125% | required only. Basic regular | | | | |
| | depending | PCR optimisation. Basic | | | | |
| | on target | regular qPCR analysis. | | | | |
| ddPCR | 0.001- | Requires more expensive | Closed | 1 | day | [31-34] |
| | 2.99% | ddPCR machine. Requires | | | | |
| | depending | probe design and | | | | |
| | on target | optimisation. More in depth | | | | |
| | and | and complicated analysis. | | | | |
| | protocol | | | | | |
| BEAMing | 0.01% | Requires very expensive | Open | ≥2 | days | [30, 39] |
| | depending | flow cytometry machine as | | | | |
| | on target | well as PCR machine. | | | | |
| | and | Requires probe design and | | | | |
| | protocol | optimisation. More in depth | | | | |
| | | and complicated analysis. | | | | |
| CAPP-seq | 0.025%- | Requires very expensive | Open | 2-5 days depending | | [40, 41] |
| (NGS) or | 0.14% | NGS machine which is also | | on the machine | | |
| standard | depending | costly to run as well as a | | | | |
| targeted deep | on target | PCR machine. CAPP-seq has | | | | |
| sequencing | and | a special protocol for sample | | | | |
| | protocol | preparation which has to be | | | | |
| | | optimised. Standard sample | | | | |
| | | preparation for targeted deep | | | | |
| | | sequencing requires large | | | | |
| | | optimisation. More in depth | | | | |
| | | and complicated analysis. | | | | |
| COLD-PCR | 0.75-3% | Requires a PCR machine. | Open or | 1-2 | days | [6, 42] |
| | depending | Requires frequent | closed | | | |
| | on target | optimisation of Tc. | depending | | | |
| | and | Depending on post PCR | on method | | | |
| | protocol | technique; additional | of | | | |
| | | equipment and optimisation | detection. | | | |
| | | may be required. | | | | |
| Enhanced- | 0.05- | Requires a pyrosequencing | Open | 2 | days | [43] |
| Ice-COLD | 0.1% | machine as well as PCR | | | | |
| PCR | | machine. Requires | | | | |
| | | optimisation of blocker and | | | | |
| | | Tc. | | | | |
| PNA LNA | 0.001- | Requires a real-time PCR | Closed | 1 | day | [44-47] |
| qPCR or | 0.01% | machine. Requires | | | | |
| LNA | | optimisation of a blocker and | | | | |
| blocking | | probe. Requires a large | | | | |
| qPCR | | number of PNA or LNA | | | | |
| (ARMS | | making it expensive. | | | | |
| qPCR) | | | | | | |
| Intplex | 0.004- | Requires a real-time PCR | Closed | 1 | day | [3] |
| (ARMS | 0.014% | machine. Requires | | | | |
| qPCR) | | optimisation of a blocker and | | | | |
| | | probe. | | | | |
| SNPase- | 0.0005% | Requires a real-time PCR | Open | 1 | day | [4] |
| ARMS qPCR | | machine. Requires expensive | | | | |
| | | SNPase enzyme. Requires | | | | |
| | | optimisation of a probe. | | | | |

TABLE 16
Demonstration of potential time savings using HOT PCR for all types of PCR. Time
savings can be increased without real-time capture or reduced with melt-curve
analysis. To simplify time saving gains; the same total cycling times have been
used for standard PCR (1 minute 30 seconds a cycle without ramping time) or fast
PCR (13 seconds a cycle without ramping time). This determines the amount of
time wasted from ramping using conventional protocols against HOT PCR protocols.

| | Total time (including | | | | | |
|---|---|---|---|---|---|---|
| | real-time capture, | | Time saving | | Time saving | |
| | excluding melt curve | | compared to | | compared to protocol | |
| Protocol | analysis) | | standard 1 | | above | |
| Standard 1 | 1 hour 51 minutes | | N/A | | N/A | |
| 95° C. 5 min × 1 | | | | | | |
| [95° C. 30 sec | | | | | | |
| 50° C. 30 sec | | | | | | |
| 72° C. 30 sec] × 40 | | | | | | |
| Standard 2 | 1 hour 45 minutes | | 6 | minutes | 6 | minutes |
| 95° C. 5 min × 1 | | | | | | |
| [95° C. 30 sec | | | | | | |
| 60° C. 30 sec | | | | | | |
| 72° C. 30 sec] × 40 | | | | | | |
| HOT Standard 1 | 1 hour 8 minutes | | 43 | minutes | 37 | minutes |
| 95° C. 5 min × 1 | | | | | | |
| [95° C. 30 sec | | | | | | |
| 71° C. 1 min] × 10 | | | | | | |
| [95° C. 30 sec | | | | | | |
| 78° C. 1 min] × 30 | | | | | | |
| HOT Standard 2 | 1 | hour | 51 | minutes | 8 | minutes |
| 95° C. 5 min × 1 | | | | | | |
| [95° C. 30 sec | | | | | | |
| 71° C. 1 min] × 2 | | | | | | |
| [95° C. 30 sec | | | | | | |
| 78° C. 1 min] × 38 | | | | | | |
| Fast 1 | 59 | minutes | 52 | minutes | 1 | minute |
| 95° C. 5 min × 1 | | | | | | |
| [95° C. 5 sec | | | | | | |
| 50° C. 5 sec | | | | | | |
| 72° C. 3 sec] × 40 | | | | | | |
| Fast 2 | 53 | minutes | 58 | minutes | 6 | minutes |
| 95° C. 5 min × 1 | | | | | | |
| [95° C. 5 sec | | | | | | |
| 60° C. 5 sec | | | | | | |
| 72° C. 3 sec] × 40 | | | | | | |

| | | | | | |
|---|---|---|---|---|---|
| HOT rapid | 26 minutes (44 minutes | 85 | minutes | 27 | minutes |
| universal | including standard | | | | |
| 95° C. 5 min × 1 | melt-curve). Universal | | | | |
| [95° C. 5 sec | conditions which | | | | |
| 66° C. 8 sec] × 10 | require no optimisation | | | | |
| [95° C. 5 sec | and give efficient | | | | |
| 70° C. 8 sec] × 30 | amplification | | | | |

| | | | |
|---|---|---|---|
| HOT rapid | 15 minutes for the | Up to 96 minutes | Up to 11 minutes |
| optimised | fastest protocol, varies | | |
| 95° C. 5 min × 1 | based on the number of | | |
| [95° C. 1 sec | cycles and primer used. | | |
| 65-72° C. 1 sec] × | | | |
| 1-20 | | | |
| [95° C. 1 sec | | | |
| 70-82° C. 1 sec] × | | | |
| 1-40 | | | |

TABLE 17
Reliable rapid amplification of DNA using HOT_PCR. Quantification if desired can be improved
with larger mastermix volumes and primer concentration optimisation and the use of probes.

| Master | | Ct: | Ct: | Ct: | Ct: | Ct: | Ct: | Ct: | Ct: | Ct: |
|---|---|---|---|---|---|---|---|---|---|---|
| mix | Target | 120 ng | 80 ng | 60 ng | 40 ng | 20 ng | 10 ng | 5 ng | 1 ng | 100 pg |
| Qiagen | KRAS | 24.74 | 24.86 | 25.31 | 25.41/25.47 | 25.62/25.73 | 27.95/27.58/ | 28.29/28.82/ | 31.09/31.18/ | |
| fast | | | | | | | 27.72 | 28.84 | 31.26 | |
| cycling | | | | | | | | | | |
| Qiagen | EGFR | 23.22 | 23.94 | 24.75 | 24.92/24.83 | 25.01/25.20 | 26.87/27.07/ | 27.81/28.57/ | 30.72/30.76/ | |
| fast | | | | | | | 27.11 | 28.28 | 30.63 | |
| cycling | | | | | | | | | | |
| SensiFAST | KRAS | 23.64 | 22.69 | 23.60 | 24.44/24.32 | 24.51/24.49 | 26.18/25.92/ | 25.46/27.00/ | 28.53/29.42/ | |
| | | | | | | | 26.69 | 27.33 | 26.03 | |
| SensiFAST | EGFR | 22.67 | 22.77 | 23.42 | 23.30/23.89 | 23.87/23.61 | 25.66/25.75/ | 26.58/26.37/ | 29.05/29.21/ | |
| | | | | | | | 25.73 | 26.15 | 29.00 | |

### REFERENCES

All references herein may be incorporated by reference.
3. Mouliere, F., et al., Circulating Cell-Free DNA from Colorectal Cancer Patients May Reveal High KRAS or BRAF Mutation Load. Translational Oncology, 2013. 6(3): p. 319-U281.
4. Stadler, J., et al., SNPase-ARMS qPCR: Ultrasensitive Mutation-Based Detection of Cell-Free Tumor DNA in Melanoma Patients. PLoS One, 2015. 10(11): p. e0142273.
6. Milbury, C. A., et al., COLD-PCR: improving the sensitivity of molecular diagnostics assays. Expert Rev Mol Diagn, 2011. 11(2): p. 159-69.
10. Newton, C. R., et al., Analysis of Any Point Mutation in DNA—the Amplification Refractory Mutation System (Arms). Nucleic Acids Research, 1989. 17(7): p. 2503-2516.
11. Thelwell, N., et al., Mode of action and application of Scorpion primers to mutation detection. Nucleic Acids Research, 2000. 28(19): p. 3752-3761.
12. Bui, M. and Z. C. Liu, Simple allele-discriminating PCR for cost-effective and rapid genotyping and mapping. Plant Methods, 2009. 5.
30. Taniguchi, K., et al., Quantitative Detection of EGFR Mutations in Circulating Tumor DNA Derived from Lung Adenocarcinomas. Clinical Cancer Research, 2011. 17(24): p. 7808-7815.
31. Sanmamed, M. F., et al., Quantitative cell-free circulating BRAFV600E mutation analysis by use of droplet digital PCR in the follow-up of patients with melanoma being treated with BRAF inhibitors. Clin Chem, 2015. 61(1): p. 297-304.
32. Abdel-Wahab, O., et al., Efficacy of intermittent combined RAF and MEK inhibition in a patient with concurrent BRAF-and NRAS-mutant malignancies. Cancer Discov, 2014. 4(5): p. 538-45.
33. Oxnard, G. R., et al., Noninvasive Detection of Response and Resistance in EGFR-Mutant Lung Cancer Using Quantitative Next-Generation Genotyping of Cell-Free Plasma DNA. Clinical Cancer Research, 2014. 20(6): p. 1698-1705.
34. Beaver, J. A., et al., Detection of Cancer DNA in Plasma of Patients with Early-Stage Breast Cancer. Clinical Cancer Research, 2014. 20(10): p. 2643-2650.
39. Diehl, F., et al., Circulating mutant DNA to assess tumor dynamics. Nat Med, 2008. 14(9): p. 985-90.
40. Newman, A. M., et al., An ultrasensitive method for quantitating circulating tumor DNA with broad patient coverage. Nat Med, 2014. 20(5): p. 548-54.
41. Dawson, S. J., et al., Analysis of circulating tumor DNA to monitor metastatic breast cancer. N Engl J Med, 2013. 368(13): p. 1199-209.
42. Hersh Abdul Ham-Karim, H. O. E., Wakkas Fadhil, Abutaleb Asiri, James Hassall and Mohammad Ilyas, COLD-HRM: A Combination of Methods to Infer the Nature of Somatic Mutations. Advances in cytology and pathology, 2017. 2(2).
43. How Kit, A., et al., Sensitive detection of KRAS mutations using enhanced-ice-COLD-PCR mutation enrichment and direct sequence identification. Hum Mutat, 2013. 34(11): p. 1568-80.
44. Guha, M., E. Castellanos-Rizaldos, and G. M. Makrigiorgos, DISSECT Method Using PNA-LNA Clamp Improves Detection of EGFR T790m Mutation. PLoS One, 2013. 8(6): p. e67782.
45. Shinozaki, M., et al., Utility of circulating B-RAF DNA mutation in serum for monitoring melanoma patients receiving biochemotherapy. Clin Cancer Res, 2007. 13(7): p. 2068-74.
46. Pinzani, P., et al., Allele specific Taqman-based real-time PCR assay to quantify circulating BRAFV600E mutated DNA in plasma of melanoma patients. Clin Chim Acta, 2010. 411(17-18): p. 1319-24.
47. Chen, D., et al., High-sensitivity PCR method for detecting BRAF V600E mutations in metastatic colorectal cancer using LNA/DNA chimeras to block wild-type alleles. Anal Bioanal Chem, 2014. 406(9-10): p. 2477-87.

1. A method of detecting one or more target nucleic acid(s) having a variant sequence in a pool of nucleic acid comprising non-variant nucleic acid and/or non-targeted variant nucleic acid, the method comprising:
providing a forward and reverse PCR primer pair capable of hybridising to the target nucleic acid having a variant sequence for PCR amplification of the target nucleic acid having a variant sequence,
wherein the terminal 3′ nucleotide of either the forward or reverse primer is arranged to form a base pair with a variant nucleotide of interest in the target nucleic acid sequence having a variant sequence, thereby forming a variant-specific primer, and
wherein the forward and reverse PCR primers have a minimum annealing temperature (Ta) of 65° C.;
carrying out a PCR in order to amplify any target nucleic acid having a variant sequence in the pool of the nucleic acid; and
detecting any PCR product or amplification in real-time, wherein the detection of a PCR product or amplification in real-time confirms the presence of the target nucleic acid having a variant sequence in the pool of the nucleic acid.

2. A method of detecting one or more target nucleic acid(s) having a variant sequence in a pool of nucleic acid that comprises non-variant nucleic acid and/or non-targeted variant nucleic acid, the method comprising:
i) providing a forward and reverse PCR primer pair capable of hybridising to the target nucleic acid having a variant sequence for PCR amplification of the target nucleic acid having a variant sequence, wherein the forward and reverse PCR primers have a minimum Ta of 65° C.;
ii) providing a blocking probe that is arranged to hybridise to the variant nucleic acid and/or non-targeted variant nucleic acid and prevent polymerisation from the forward and/or reverse PCR primers;
carrying out a PCR in order to amplify any target nucleic acid having a variant sequence in the pool of the nucleic acid; and
detecting any PCR product or amplification in real-time, wherein the detection of a PCR product or amplification in real-time confirms the presence of the target nucleic acid having a variant sequence in the pool of the nucleic acid.

3. A method of highly specific polymerase chain reaction (PCR) amplification of one or more target nucleic acids in a pool of nucleic acid, the method comprising:
providing a forward and reverse PCR primer pair capable of hybridising to the target nucleic acid for PCR amplification of the target nucleic acid, wherein the forward and reverse PCR primers each comprise a 5′ tag of non-complementary nucleotides and have a minimum annealing temperature (Ta) of 65° C.;
carrying out a PCR with one or more first cycle temperature profiles, such that the 5′ tag of non-complementary nucleotides of the forward and reverse PCR primer pair become incorporated into the PCR amplicons,
wherein the first cycle temperature profile provides an annealing temperature of at least 65° C. and which is suitable for the annealing of the forward and reverser primer pair such that they hybridise to the target nucleic acid, and
carrying out a PCR with one or more second cycle temperature profiles in order to amplify the PCR amplicons from the PCR cycle(s) of the first cycle temperature profile,
wherein the second cycle temperature profile provides an annealing temperature that is higher than the first cycle temperature profile and which is suitable for the annealing of the PCR amplicons.

4. The method according to claim 1 or 2, wherein carrying out the PCR comprises the method of highly specific PCR in accordance with claim 3.

5. The method according to any preceding claim, wherein the forward and reverse PCR primers each comprise a 5′ tag of non-complementary nucleotides.

6. The method according to any preceding claim, wherein the forward and reverse PCR primers each comprise nucleotide analogues.

7. The method according to any of claim 1, 2, or 4-6, wherein the variant sequence is a mutation, and the non-variant nucleic acid is wild-type nucleic acid.

8. The method according to any of claim 1, 2, or 4-7, wherein the variation comprises a single nucleotide variation (SNV).

9. The method according to any of claim 1, 2, or 4-8, wherein the variation comprises a nucleotide deletion, insertion, amplification or rearrangement.

10. The method according to any preceding claim, wherein the forward and/or reverse primer comprise nucleotide analogue selected from LNA, BNA or PNA.

11. The method according to any preceding claim, wherein the forward or the reverse primer comprises a nucleotide analogue, such as LNA, PNA, or BNA, at the terminal 3′ position.

12. The method according to any preceding claim, wherein the forward or the reverse primer comprises a nucleotide analogue, such as LNA, PNA, or BNA, at the terminal 3′ position, wherein the remaining primer sequence comprises DNA.

13. The method according to any preceding claim, wherein the forward or the reverse primer comprises a nucleotide analogue, such as LNA, PNA, or BNA, at the terminal 3′ position, and a 5′ tag of non-complementary nucleotides.

14. The method according to any preceding claim, wherein the forward and/or reverse primers comprise a GC content of between about 40% and about 60%.

15. The method according to any of claims 5-14, wherein the 5′ tag of non-complementary nucleotides consists of a sequence of between about 5 and 100 nucleotides.

16. The method according to any of claims 5-15, wherein the GC content of the 5′ tag is 100%.

17. The method according to any of claims 5-16, wherein the 5′ tag of non-complementary nucleotides comprises or consists of a sequence of 5′-gggccggccc-3′ (SEQ ID NO: 35) or 5′-gggccgggccggccc-3′ (SEQ ID NO: 36).

18. The method according to any preceding claim, wherein the amplification of the PCR product is detected during the PCR, using Real-Time PCR.

19. A method of determining the status of a condition associated with a known mutation in a subject, the method comprising:
providing a sample from the subject comprising a target nucleic acid, wherein the target nucleic acid may contain the mutation;
detecting the mutation in the sample in accordance with the method of any of claim 1, 2, or 4-18,
wherein the detection of the mutation is indicative of the status of the condition associated with the mutation in the subject.

20. The method according to claim 15, wherein the condition comprises cancer.

21. A primer for use in a primer pair for detecting a target nucleic acid having a variant sequence in a pool of nucleic acid, wherein the primer is capable of hybridising to the target nucleic acid having a variant sequence for PCR amplification of the target nucleic acid having a variant sequence,
wherein the terminal 3′ nucleotide of the primer is arranged to base pair with the variant nucleotide of interest in the target nucleic acid having a variant sequence;
wherein the primer comprises a 5′ tag of non-complementary nucleotides and/or comprise one or more nucleotide analogues such that the primers has a minimum Ta of 65° C.

22. The primer according to claim 21, wherein the primer comprises a nucleotide analogue at the terminal 3′ position.

23. The primer according to claim 21 or 22, wherein the primer comprises a nucleotide analogue at the terminal 3′ position, wherein the remaining primer sequence comprises DNA.

24. The primer according to any of claims 21-23, wherein the primer comprises a nucleotide analogue at the terminal 3′ position and a 5′ tag of non-complementary nucleotides.

25. A forward and reverse primer pair for the PCR detection of a KRAS, PIK3CA, EGFR, APC or BRAF mutation in a target nucleic acid, wherein the forward and/or reverse primer is selected from the KRAS, PIK3CA, EGFR, APC or BRAF forward and reverse primers respectively of table 1 herein
wherein the forward and reverse primers further comprise a 5′ tag of nucleotides that are non-complementary to the target nucleic acid, and/or wherein terminal 3′ nucleotide of the forward or reverse primer is substituted with a nucleotide analogue.

26. A composition comprising the primer according to any of claims 21-24 or primer pair according to claim 25; and optionally a blocking probe.

27. A kit comprising the primer according to any of claims 21-24 or primer pair according to claim 25, or the composition according to claim 26.

28. The kit according to claim 27, further comprising a polymerase and/or a blocking probe.

29. Use of the primer according to any of claims 21-24 or primer pair according to claim 25, or the composition according to claim 26, or the kit in accordance with claim 26 or 27, for the detection of a target nucleic acid having a variant sequence in a pool of nucleic acid.

30. The use according to claim 29 for diagnosis or prognosis of a condition or response to chemotherapy associated with a mutation, in a subject.
